SEQUENCE LISTING (20 sequences)

SEQ ID NO: 1 - protein, 180 aa, Felis sp.
MKSIYFVAGLFVMLVQGSWQRSLQDTEEKSRSFPAPQTDPLNDPDQMIEDKRHSQGTFTSDYSKYLDSRRAQDFVQWLMNTKRNKNNIAKRHDEFERHAEGTFTSDVSSYLEGQAAKEFIAWLVKGRGRRDFPEEVTIVEELRRRHADGSFSDEMNTVLDNLATRDFINWLLQTKITERK

SEQ ID NO: 2 - protein, 37 aa, Felis sp.
HDEFERHAEGTFTSDVSSYLEGQAAKEFIAWLVKGRG

SEQ ID NO: 3 - protein, 31 aa, Felis sp.
HAEGTFTSDVSSYLEGQAAKEFIAWLVKGRG

SEQ ID NO: 4 - protein, 31 aa, Synthetic construct
HGEGTFTSDVSSYLEEQAAKEFIAWLVKGGG

SEQ ID NO: 5 - protein, 237 aa, Felis sp.
ARKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 6 - protein, 15 aa, Synthetic construct
GGGGSGGGGSGGGGS

SEQ ID NO: 7 - protein, 19 aa, Felis sp.
QHVFLAPQQALSLLQRVRR

SEQ ID NO: 8 - protein, 50 aa, Synthetic construct
QHVFLAPQQALSLLQRVRRHGEGTFTSDVSSYLEEQAAKEFIAWLVKGGG

SEQ ID NO: 9 - protein, 302 aa, Synthetic construct
QHVFLAPQQALSLLQRVRRHGEGTFTSDVSSYLEEQAAKEFIAWLVKGGGGGGGSGGGGSGGGGSARKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPG

SEQ ID NO: 10 - protein, 224 aa, Felis sp.
DCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPG

SEQ ID NO: 11 - protein, 289 aa, Synthetic construct
QHVFLAPQQALSLLQRVRRHGEGTFTSDVSSYLEEQAAKEFIAWLVKGGGGGGGSGGGGSGGGGSDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPG

SEQ ID NO: 12 - protein, 270 aa, Synthetic construct
HGEGTFTSDVSSYLEEQAAKEFIAWLVKGGGGGGGSGGGGSGGGGSDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPG

SEQ ID NO: 13 - protein, 19 aa, Synthetic construct
QHVFLAPQQALSLLQRVHD

SEQ ID NO: 14 - protein, 50 aa, Synthetic construct
QHVFLAPQQALSLLQRVHDHGEGTFTSDVSSYLEEQAAKEFIAWLVKGGG

SEQ ID NO: 15 - protein, 224 aa, Synthetic construct
DCPKCPPPEMLGGPSIFIFPPKPKDTLSISRTPEVTCLVVDLGPDDSDVQITWFVDNTQVYTAKTSPREEQFSSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPG

SEQ ID NO: 16 - protein, 289 aa, Synthetic construct
QHVFLAPQQALSLLQRVHDHGEGTFTSDVSSYLEEQAAKEFIAWLVKGGGGGGGSGGGGSGGGGSDCPKCPPPEMLGGPSIFIFPPKPKDTLSISRTPEVTCLVVDLGPDDSDVQITWFVDNTQVYTAKTSPREEQFSSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPG

SEQ ID NO: 17 - protein, 289 aa, Synthetic construct
QHVFLAPQQALSLLQRVRRHGEGTFTSDVSSYLEEQAAKEFIAWLVKGGGGGGGSGGGGSGGGGSDCPKCPPPEMLGGPSIFIFPPKPKDTLSISRTPEVTCLVVDLGPDDSDVQITWFVDNTQVYTAKTSPREEQFSSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPG

SEQ ID NO: 18 - protein, 224 aa, Synthetic construct
DCPKCPPPEMLGGPSIFIFPPKPKDTLSISRTPEVTCLVVDLGPDDSDVQITWFVDNTQVYTAKTSPREEQFQSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPG

SEQ ID NO: 19 - protein, 289 aa, Synthetic construct
QHVFLAPQQALSLLQRVRRHGEGTFTSDVSSYLEEQAAKEFIAWLVKGGGGGGGSGGGGSGGGGSDCPKCPPPEMLGGPSIFIFPPKPKDTLSISRTPEVTCLVVDLGPDDSDVQITWFVDNTQVYTAKTSPREEQFQSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPG

SEQ ID NO: 20 - protein, 289 aa, Synthetic construct
QHVFLAPQQALSLLQRVHDHGEGTFTSDVSSYLEEQAAKEFIAWLVKGGGGGGGSGGGGSGGGGSDCPKCPPPEMLGGPSIFIFPPKPKDTLSISRTPEVTCLVVDLGPDDSDVQITWFVDNTQVYTAKTSPREEQFQSTYRVVSVLPILHQDWLKGKEFKCKVNSKSLPSPIERTISKDKGQPHEPQVYVLPPAQEELSRNKVSVTCLIEGFYPSDIAVEWEITGQPEPENNYRTTPPQLDSDGTYFLYSRLSVDRSRWQRGNTYTCSVSHEALHSHHTQKSLTQSPG

(19) United States
(12) Patent Application Publication
(10) Pub. No.: US 2026/0201009 A1
(43) Pub. Date: Jul. 16, 2026

(54) FC FUSION PROTEIN THERAPEUTICS FOR COMPANION ANIMAL OBESITY AND METHODS OF USE

(71) Applicant: AKSTON BIOSCIENCES CORPORATION, Beverly, MA (US)

(72) Inventors: Todd C. ZION, Salem, MA (US); Thomas M. LANCASTER, Wenham, MA (US)

(21) Appl. No.: 19/633,161

(22) Filed: Mar. 30, 2026

Related U.S. Application Data
(63) Continuation of application No. PCT/US2025/041488, filed on Aug. 11, 2025.
(60) Provisional application No. 63/682,095, filed on Aug. 12, 2024.
(60) Provisional application No. 63/682,091, filed on Aug. 12, 2024.

(51) Int. Cl. C07K 14/605; A61K 38/00; A61P 3/04

(52) U.S. Cl. CPC C07K 14/605; A61P 3/04; A61K 38/00; C07K 2319/30

(57) ABSTRACT

The present disclosure provides recombinantly manufactured fusion proteins comprising a GLP-1 protein fragment or an analog thereof linked to a feline Fc fragment. Embodiments include the administration of the fusion proteins to patients and their use as an appetite suppressant, for treatment of obesity, for prevention of obesity, or for treatment of other conditions associated with excess weight in cats. Exemplary Fc fusion proteins and pharmaceutical formulations of exemplary Fc fusion proteins are provided, in addition to methods of use and preparation.

### CROSS-REFERENCE TO RELATED APPLICATIONS

The present application is a continuation of International Patent Application Serial No. PCT/US2025/041488, filed Aug. 11, 2025, which claims the priority benefit of U.S. Provisional Patent Application Ser. No. 63/682,091, filed Aug. 12, 2024, and U.S. Provisional Patent Application Ser. No. 63/682,095, filed Aug. 12, 2024, each entitled “FC FUSION PROTEIN THERAPEUTICS FOR COMPANION ANIMAL OBESITY AND METHODS OF USE,” and each incorporated by reference in its entirety herein.

### SEQUENCE LISTING

The following application contains a sequence listing filed electronically as a Standard ST.26 compliant XML file entitled “ABC-072US.xml,” created on Aug. 6, 2025, as 21,516 bytes in size, the entire contents of which are incorporated by reference herein.

### TECHNICAL FIELD

The present technology relates to fusion proteins comprising a Glucagon Like Peptide-1 (GLP-1) protein or an analog thereof linked to Fc fragments and their use as an appetite suppressant, for treatment of obesity, for prevention of obesity, or for treatment of other conditions associated with excess weight in cats.

### BACKGROUND

The following description of the background is provided simply as an aid in understanding the present technology and is not admitted to describe or constitute prior art to the present technology.

### Feline Obesity

Approximately 11-63% of domestic cats are affected by obesity (Michel K and Scherk M. From problem to success: feline weight loss programs that work. J. Feline Med Surg 2012; 14: 327-336). Feline obesity is characterized as excess adipose tissue accumulation. (German A J. The growing problem of obesity in dogs and cats. J. Nutr 2006; 136 Suppl 7: 1940-1946). Treatment for feline obesity treatment may include veterinary therapeutic diets for weight loss, feeding management strategies and exercise, however it is a slow and often unsuccessful process. (Godfrey et al (2024) Identifying the target population and preventive strategies to combat feline obesity. J Feline Med Surg. 2024 February; 26(2): 1098612X241228042). Of the cats that do lose weight, more than half regain an appreciable amount of the weight lost. (Deagle G, et al. Long-term follow-up after weight management in obese cats. J Nutr Sci 2014; 3: e25. DOI: 10.10171jns.2014.36). Obesity is the most common nutritional disorder in cats within general clinical practice and a 2009 report estimated that the disease costs obese cat owners more than US$1000 in veterinary bills in just the first 12 months after the cats are diagnosed with obesity, due to obesity-associated comorbidities. (Bomberg E, et al. (2017) The financial costs, behaviour and psychology of obesity: a one health analysis. J Comp Pathol 2017; 156: 310-325).

Major risk factors for feline obesity include age, sex, breed, indoor confinement, feeding primarily a dry food diet, free-feeding and feeding frequency, overestimating food allotments, neutering, and owner (e.g., providing treats).

In consequence, there is a need for a long-acting treatment for obesity in cats that can be used as an appetite suppressant either as a preventative strategy to keep weight at an optimum level, a therapy administered to cause weight loss, or as an adjunct therapy to prevent a recurrence of feline obesity after weight has been lost.

### Proglucagon and Glucagon

Proglucagon is a 180 amino acid prepropolypeptide expressed in the alpha cells a of the pancreas, in the intestinal L cells in the distal ileum and colon and also in some neurons of the central nervous system (CNS), more specifically in solitary tract nucleus (Irwin. D. I. (2021). Variation in the Evolution and Sequences of Proglucagon and the Receptors for Proglucagon-Derived Peptides in Mammals. Front. Endocrinol. 12; Model. et al. (2022). Physiological and pharmacological actions of glucagon like peptide-1 (GLP-1) in domestic animals. Veterinary and Animal Science. 6: 100245; Müller. T. D. et al. (2019). Glucagon-like peptide 1 (GLP-1). Molecular Metabolism, 30, 72-130). The feline proglucagon sequence (NCBI Reference Sequence: XM 019838170.3 is shown as SEQ ID NO: 1.

(SEQ ID NO: 1)
MKSIYFVAGLFVMLVQGSWQRSLQDTEEKSRSFPAPQTDPLNDPDQMI
EDKRHSQGTFTSDYSKYLDSRRAQDFVQWLMNTKRNKNNIAKRHDEFE
RHAEGTFTSDVSSYLEGQAAKEFIAWLVKGRGRRDFPEEVTIVEELRR
RHADGSFSDEMNTVLDNLATRDFINWLLQTKITERK.

The proglucagon protein is cleaved into different secretin hormones including glucagon and GLP-1. The primary function of glucagon is to act as the counter regulatory hormone to insulin with a major role in stimulating the production and release of glucose from the liver when blood glucose levels are low (Irwin. D. L (2021). Variation in the Evolution and Sequences of Proglucagon and the Receptors for Proglucagon-Derived Peptides in Mammals. Front. Endocrinol. 12). Glucagon is selectively cleaved from proglucagon by tissue-specific proteolytic processing of the proglucagon by prohormone convertase enzymes resulting in the production of glucagon as the major product secreted from pancreatic α-cells. Glucagon is secreted from the α-cells of the pancreas in response to low blood sugar, with the main target organ for glucagon being the liver. Glucagon stimulates glycogen breakdown and inhibits glycogen biosynthesis. It also inhibits fatty acid synthesis but enhances gluconeogenesis. The net result of these actions is to significantly increase the release of glucose to the liver.

### Glucagon Like Peptide 1 (GLP-1)

Glucagon Like Peptide 1 (GLP-1) is an endogenous peptide hormone found in mammals. GLP-1 is derived from the proglucagon protein.

GLP-1 is synthesized by intestinal “L” cells, which are located mainly in the most distal intestinal regions, such as the ileum and colon. As with glucagon, GLP-1 is also derived from the proglucagon protein but from a different section of the protein sequence. Proglucagon is processed to form GLP-1 (Irwin. D. M and Wong. J. (1995) Trout and Chicken Proglucagon: Alternative Splicing Generates mRNA Transcripts Encoding Glucagon-Like Peptide 2. Mol Endocrinol 9:267-77; and Bell et al. (1983). Exon duplication and divergence in the human preproglucagon gene, Nature 304:368-371). GLP-1 is initially an inactive 37 amino acid propeptide (SEQ ID NO: 2) formed from the cleavage of proglucagon. The native GLP-1(1-37) SEQ ID NO: 2 is shown below:

(SEQ ID NO: 2)
HDEFERHAEGTFTSDVSSYLEGQAAKEFIAWLVKGRG.

By convention, the numbering of the amino acids of GLP-1 is based on the GLP-1(1-37) formed from cleavage of proglucagon. However, this GLP-1 is biologically inactive. There are two recognized active forms of GLP-1: GLP-1(7-37) (shown as SEQ ID NO: 3 below) and GLP-1(7-36)NH₂. The biologically active forms are generated from further processing of the GLP-1(1-37) of SEQ ID NO: 2, which following the numbering convention, yields GLP-1(7-37) and GLP-1(7-36)-NH₂. Both biologically active forms involve the removal of 6 amino acids from the amino-terminus of the GLP-1(1-37) of SEQ ID NO: 2. The first biologically active form is the native feline GLP-1(7-37) of SEQ ID NO: 3 which is shown below:

(SEQ ID NO: 3)
HAEGTFTSDVSSYLEGQAAKEFIAWLVKGRG.

The positions of the GLP-1(1-37) and GLP-1(7-37) within the proglucagon sequence are shown in a Clustal Omega sequence comparison shown in FIG. 2.

In this second biologically active form, the C-terminal glycine at position 37 is transformed to an amide in vivo to form GLP-1(7-36)NH₂ (GLP-1(7-36) amide). GLP-1(7-37) and GLP-1(7-36)NH₂ are insulinotropic hormones of equal potency. The most common form of GLP-1 that circulates in the body is GLP-1(7-36)NH₂. For convenience, the term “GLP-1” or “GLP-1(7-37)”, is used to refer to both of these forms.

The amino acid sequence of GLP-1 is 100% homologous in all mammals studied so far, implying a critical physiological role. In response to stimulation by intraluminal glucose, GLP-1 can increase the feeling of satiety (fullness after eating).

The plasma half-life of active GLP-1 is less than 5 minutes, and its metabolic clearance rate is around 12-13 minutes (Holst. J. J. (1994). Glucagonlike peptide 1: A newly discovered gastrointestinal hormone. Gastroenterology, 107:1848-1855, 1994). Active GLP-1 is degraded by the enzymes dipeptidyl peptidase (DPP-IV, also known as CD26) and neutral endopeptidase 24.11 (NEP-24.11) into inactive forms. The major protease involved in the metabolism of GLP-1 is DPP-IV which cleaves the N-terminal His-Ala dipeptide, producing metabolites, GLP-1(9-37) or GLP-1(9-36)NH₂ which are variously described as inactive, weak agonist or antagonists of GLP-1 receptor. DPP-IV and NEP are ubiquitous in tissues, and DPP-IV is also present in soluble form in the blood. Consequently, when injected intravenously, active GLP-1 is quickly degraded into inactive forms. This results in a short structural half-life for GLP-1 (1-2 mins) when administered intravenously.

GLP-1 treatment has many beneficial effects, but it has a very short structural half-life which prevents its widespread use in the clinical setting. This has led to the development of drugs that incorporate DPP-IV inhibitors to prolong the structural half-life of GLP-1. These drugs are known as GLP-1 agonists. GLP-1 agonists are used as medications in humans to treat obesity, as they can cause weight loss as they slow down food digestion and reduce appetite. Examples of these long-acting, DPP-IV-resistant, synthetic GLP-1 receptor agonists for use in human medications are Semaglutide (Ozempic®), Dulaglutide (Trulicity®) and Exenatide (Byetta®). Other attempts have been made to prevent DPP-IV enzymatic degradation of GLP-1 peptides by linking additional peptides to the N-terminal side of the GLP-1 peptides. (Deacon C F, Knudsen L B, Madsen K, et al. Dipeptidyl peptidase IV resistant analogues of glucagon-like peptide-1 which have extended metabolic stability and improved biological activity. Diabetologia. 1998 March; 41(3):271-278. DOI: 10.1007 s001250050903. PMID: 9541166.)

### GLP-1 Receptor (GLP-1R)

GLP-1 receptors are expressed in several brainstem nuclei involved in appetite regulation. The GLP-1 receptor (GLP-1R) is a G protein coupled receptor of 463 amino acids and is localized in pancreatic beta cells, in the lungs and to a lesser extent in the brain, adipose tissue and kidneys. The stimulation of GLP-1R by GLP-1(7-37) or GLP-1(7-36)NH2 results in adenylate cyclase activation, cAMP synthesis, membrane depolarization, and rise in intracellular calcium.

When GLP-1 binds to its receptor, it activates several pathways that influence appetite and satiety. In turn, the GLP-1R activation affects brain regions involved in appetite and reward, such as the amygdala, hypothalamus, and insula (Barakat. G. (2024) Satiety: a gut-brain-relationship The Journal of Physiological Sciences 74: Article 11). This modulation helps reduce the desire to eat and increases feelings of fullness. The GLP-1R activation slows down gastric emptying, meaning food stays in the stomach longer. This delay contributes to prolonged feelings of fullness and reduced hunger. GLP-1 also influences other hormones related to hunger and satiety, such as insulin and glucagon, further promoting a sense of fullness and reducing food intake. These combined effects make GLP-1 receptor agonists a target for treatments aimed at weight management and obesity.

### Fc Fusion Proteins

An Fc fusion protein is comprised of a species-specific immunoglobin Fc domain that is linked to another peptide such as a protein or peptide with therapeutic potential. As used herein, the terms “fusion protein” and “Fc fusion protein” generally mean a protein comprising more than one part, for example from different sources (e.g., different proteins, polypeptides, cells, etc.), that are covalently linked through peptide bonds. Fc fusion proteins are preferably covalently linked by (i) connecting the genes that encode for each part into a single nucleic acid molecule and (ii) expressing in a host cell (e.g., HEK cell or CHO cell) the protein for which the nucleic acid molecule encodes. The fully recombinant synthesis approach is preferred over methods in which the therapeutic protein and Fc fragments are synthesized separately and then chemically conjugated. The chemical conjugation step and subsequent purification process increase the manufacturing complexity, reduce product yield, and increase cost.

The terms “Fc fragment”, “Fc region”, “Fc domain”, or “Fc polypeptide” are used herein to define a C-terminal region of an immunoglobulin heavy chain. The Fc fragment, region, domain, or polypeptide may be a native sequence Fc region or a variant/mutant Fc region. Although the boundaries of the Fc region of an immunoglobulin heavy chain may vary, they generally comprise some or all of the hinge region of the heavy chain, the CH2 region of the heavy chain, and the CH3 region of the heavy chain. The hinge region of an Fc fragment comprises amino acid sequences that connect the CH1 domain of the heavy chain to the CH2 region of the heavy chain and contains one or more cysteines that form one or more interheavy chain disulfide bonds to form a homodimer of an Fc fusion protein from two identical but separate monomers of the Fc fusion protein. The hinge region may comprise all or part of a naturally occurring amino acid sequence or a non-naturally occurring amino acid sequence.

The presence of the Fc domain increases the plasma pharmacokinetic half-life due to its interaction with the neonatal Fc-receptor (FcRn) in addition to slower renal clearance of the Fc fusion protein due to the large molecule size, resulting in in vivo recycling of the molecule achieving prolonged activity of the linked peptide and improved solubility and stability of the Fc fusion protein molecule. The Fc domain also enables Fc fusion proteins to interact with Fc receptors on immune cells. In some examples, the therapeutic protein or peptide is linked to the immunoglobin Fc domain via a linker. The therapeutic protein or peptide and linker replace the variable region of an antibody while keeping the Fc region intact.

An Fc receptor (FcR) generally means a receptor that binds to an Fc fragment or to the Fc region of an antibody. In examples, the FcR is a native sequence of a mammalian FcR, and the FcR is one which binds an Fc fragment or the Fc region of an IgG antibody (a gamma receptor) and includes without limitation, receptors of the Fc(gamma) receptor I, Fc(gamma) receptor Ia, Fc(gamma) receptor IIb, and Fc(gamma) receptor III subclasses (and their species-specific equivalents, e.g., feline-specific equivalents or feline-specific equivalents), including allelic variants and alternatively spliced forms of these receptors. “FcR” also includes the neonatal receptor, FcRn, which is responsible for the transfer of maternal IgG molecules to the fetus and is also responsible for the prolonged in vivo elimination half-lives of antibodies and Fc-fusion proteins in vivo. In examples, FcR of human origin are used in vitro (e.g., in an assay) to measure the binding of Fc fusion proteins comprising Fc fragments of any mammalian origin so as to assess their FcR binding properties. Those skilled in the art will understand that mammalian FcR from one species (e.g., FcR of human origin) are sometimes capable of in vitro binding of Fc fragments from a second species (e.g., FcR of feline origin). Without being bound to any particular theory, one possible cause of the generation of anti-drug antibodies and the reduction in bioactivity is increased interaction of the Fc fragment of an Fc fusion protein with various receptors of the immune system (e.g., Fc(gamma) receptors, e.g., Fc(gamma)R).

### SUMMARY OF THE PRESENT TECHNOLOGY

Described herein are fusion proteins, each comprising a respective Glucagon Like Peptide 1 (GLP-1) analog (GLP-1-analog) and an Fc fragment mutated to prevent glycosylation, wherein the GLP-1-analog and the Fc fragment are connected by a peptide linker. In one or more embodiments, the GLP-1-analog comprises a GLP-1 fragment comprising a functional fragment, analog, or variant/mutant thereof. In one or more embodiments the GLP-1-analog-Fc fusion protein comprises a respective feline thrombin signal peptide linked to a respective Glucagon Like Peptide 1 (GLP-1) protein fragment (collectively, a GLP-1-analog) and an Fc fragment, wherein the GLP-1-analog and the Fc fragment are connected by a peptide linker.

In one or more embodiments, the GLP-1-analog comprises a feline thrombin signal peptide QHVFLAPQQALSLLQRVRR (SEQ ID NO: 7) combined with a GLP-1 fragment of HGEGTFTSDVSSYLEEQAAKEFIAWLVKGGG (SEQ ID NO: 4), or a functional fragment, analog, or variant/mutant thereof. In one or more embodiments, the GLP-1-analog comprises a sequence of SEQ ID NO: 8, or a functional fragment, analog, or variant/mutant thereof.

(SEQ ID NO: 8)
QHVFLAPQQALSLLQRVRRHGEGTFTSDVSSYLEEQAAKEFIAW
LVKGGG.

In one or more embodiments, the GLP-1-analog comprises a feline thrombin signal peptide QHVFLAPQQALSLLQRVHD (SEQ ID NO: 13) combined with a GLP-1 fragment of HGEGTFTSDVSSYLEEQAAKEFIAWLVKGGG (SEQ ID NO: 4), or a functional fragment, analog, or variant/mutant thereof. In one or more embodiments, the GLP-1-analog comprises a sequence of SEQ ID NO: 14, or a functional fragment, analog, or variant/mutant thereof.

(SEQ ID NO: 14)
QHVFLAPQQALSLLQRVHDHGEGTFTSDVSSYLEEQAAKEFIAW
LVKGGG.

In one or more embodiments, the Fc fragment is mutated to prevent glycosylation. In one or more embodiments, the mutation to the Fc fragment to prevent glycosylation comprises a mutation of the natural glycosylation site of the Fc fragment, at the 73rd amino acid from the N-terminus of the native feline IgG1b Fc fragment of SEQ ID NO: 10, from asparagine (N) to serine (S) or glutamine (Q).

In one or more embodiments, the Fc fragment mutated to prevent glycosylation comprises a sequence or functional fragment of SEQ ID NO: 18.

(SEQ ID NO: 18)
DCPKCPPPEMLGGPSIFIFPPKPKDTLSISRTPEVTCLVVDLGP
DDSDVQITWFVDNTQVYTAKTSPREEQFQSTYRVVSVLPILHQD
WLKGKEFKCKVNSKSLPSPIERTISKDKGQPHEPQVYVLPPAQE
ELSRNKVSVTCLIEGFYPSDIAVEWEITGQPEPENNYRTTPPQL
DSDGTYFLYSRLSVDRSRWQRGNTYTCSVSHEALHSHHTQKSLT
QSPG.

In one or more embodiments, the Fc fragment mutated to prevent glycosylation comprises a sequence or functional fragment of SEQ ID NO: 15.

(SEQ ID NO: 15)
DCPKCPPPEMLGGPSIFIFPPKPKDTLSISRTPEVTCLVVDLGP
DDSDVQITWFVDNTQVYTAKTSPREEQFSSTYRVVSVLPILHQD
WLKGKEFKCKVNSKSLPSPIERTISKDKGQPHEPQVYVLPPAQE
ELSRNKVSVTCLIEGFYPSDIAVEWEITGQPEPENNYRTTPPQL
DSDGTYFLYSRLSVDRSRWQRGNTYTCSVSHEALHSHHTQKSLT
QSPG.

In one or more embodiments, the present disclosure provides a fusion protein comprising a GLP-1-analog and an Fc fragment, wherein the GLP-1-analog and the Fc fragment are connected by a linker (e.g., peptide linker) comprising the sequence of SEQ ID NO: 6.

(SEQ ID NO: 6)
GGGGSGGGGSGGGGS.

In an embodiment, the present disclosure provides for a fusion protein comprising a GLP-1-analog and an Fc fragment, wherein the GLP-1-analog and the Fc fragment are connected by a linker (e.g., a peptide linker), and wherein the GLP-1-analog-Fc fusion protein comprises the sequence of SEQ ID NO: 19.

(SEQ ID NO: 19)
QHVFLAPQQALSLLQRVRRHGEGTFTSDVSSYLEEQAAKEFIAW
LVKGGGGGGGSGGGGSGGGGSDCPKCPPPEMLGGPSIFIFPPKP
KDTLSISRTPEVTCLVVDLGPDDSDVQITWFVDNTQVYTAKTSP
REEQFQSTYRVVSVLPILHQDWLKGKEFKCKVNSKSLPSPIERT
ISKDKGQPHEPQVYVLPPAQEELSRNKVSVTCLIEGFYPSDIAV
EWEITGQPEPENNYRTTPPQLDSDGTYFLYSRLSVDRSRWQRGN
TYTCSVSHEALHSHHTQKSLTQSPG.

In an embodiment, the present disclosure provides for a fusion protein comprising a GLP-1-analog and an Fc fragment, wherein the GLP-1-analog and the Fc fragment are connected by a linker (e.g., a peptide linker), and wherein the GLP-1-analog-Fc fusion protein comprises the sequence of SEQ ID NO: 17.

(SEQ ID NO: 17)
QHVFLAPQQALSLLQRVRRHGEGTFTSDVSSYLEEQAAKEFIAW
LVKGGGGGGGSGGGGSGGGGSDCPKCPPPEMLGGPSIFIFPPKP
KDTLSISRTPEVTCLVVDLGPDDSDVQITWFVDNTQVYTAKTSP
REEQFSSTYRVVSVLPILHQDWLKGKEFKCKVNSKSLPSPIERT
ISKDKGQPHEPQVYVLPPAQEELSRNKVSVTCLIEGFYPSDIAV
EWEITGQPEPENNYRTTPPQLDSDGTYFLYSRLSVDRSRWQRGN
TYTCSVSHEALHSHHTQKSLTQSPG.

In an embodiment, the present disclosure provides for a fusion protein comprising a GLP-1-analog and an Fc fragment, wherein the GLP-1-analog and the Fc fragment are connected by a linker (e.g., a peptide linker), and wherein the GLP-1-analog-Fc fusion protein comprises the sequence of SEQ ID NO: 20.

(SEQ ID NO: 20)
QHVFLAPQQALSLLQRVHDHGEGTFTSDVSSYLEEQAAKEFIAW
LVKGGGGGGGSGGGGSGGGGSDCPKCPPPEMLGGPSIFIFPPKP
KDTLSISRTPEVTCLVVDLGPDDSDVQITWFVDNTQVYTAKTSP
REEQFQSTYRVVSVLPILHQDWLKGKEFKCKVNSKSLPSPIERT
ISKDKGQPHEPQVYVLPPAQEELSRNKVSVTCLIEGFYPSDIAV
EWEITGQPEPENNYRTTPPQLDSDGTYFLYSRLSVDRSRWQRGN
TYTCSVSHEALHSHHTQKSLTQSPG.

In an embodiment, the present disclosure provides for a fusion protein comprising a GLP-1-analog and an Fc fragment, wherein the GLP-1-analog and the Fc fragment are connected by a linker (e.g., a peptide linker), and wherein the GLP-1-analog-Fc fusion protein comprises the sequence of SEQ ID NO: 16.

(SEQ ID NO: 16)
QHVFLAPQQALSLLQRVHDHGEGTFTSDVSSYLEEQAAKEFIAW
LVKGGGGGGGSGGGGSGGGGSDCPKCPPPEMLGGPSIFIFPPKP
KDTLSISRTPEVTCLVVDLGPDDSDVQITWFVDNTQVYTAKTSP
REEQFSSTYRVVSVLPILHQDWLKGKEFKCKVNSKSLPSPIERT
ISKDKGQPHEPQVYVLPPAQEELSRNKVSVTCLIEGFYPSDIAV
EWEITGQPEPENNYRTTPPQLDSDGTYFLYSRLSVDRSRWQRGN
TYTCSVSHEALHSHHTQKSLTQSPG.

In embodiments, a fusion protein of the present disclosure comprises a dimer, wherein the dimer comprises two identical monomers bound together via disulfide bonds e.g., the fusion protein is a homodimer. In embodiments, the percentage homodimer of the GLP-1-analog-Fc fusion protein is greater than or equal to 85%.

In embodiments, the fusion proteins described herein are made using CHO cells, and the resulting manufacturing titer after purification using Protein A beads or a Protein A column is greater than 300 mg/L. In embodiments, the GLP-1 receptor binding OD450 at a concentration of 2000 ng/mL for the GLP-1-analog Fc fusion proteins described herein is greater than or equal to 0.25, as measured in a GLP-1 receptor binding assay. In embodiments, the GLP-1 receptor binding EC50 for the fusion proteins described herein is greater than or equal to 250 ng/mL, as measured in a GLP-1 receptor binding assay.

In embodiments, the serum pharmacokinetic half-life of the fusion proteins described herein in the blood or serum of a target animal upon administration is longer than about 24 hours (1 day), 36 hours (1.5 days), 48 hours (2 days), 52.8 hours (2.2 days), 60 hours (2.5 days), or 72 hours (3 days).

In aspects, a method is described for lowering the body weight of a cat, for treating obesity in a cat, for preventing obesity in a cat, or for treating other conditions associated with excess weight in cats, the method comprising administering a therapeutically effective amount of a fusion protein as described herein or a pharmaceutical composition thereof to the cat. In some embodiments, the pharmaceutical composition comprises a composition comprising a fusion protein as described herein and a pharmaceutically acceptable carrier. In some embodiments, the cat is overweight or obese. In some embodiments the cat is not obese or is no longer obese and the fusion protein is used to prevent weight gain. In some embodiments, the composition is administered via injection. In some embodiments, the composition is administered subcutaneously or intramuscularly. In some embodiments, the composition is administered daily, twice weekly, once weekly or once biweekly to the cat.

In aspects, a cell is engineered to express a fusion protein as described herein. In examples, the cell is transfected with a nucleic acid encoding the fusion protein. In examples, the cell is a CHO cell. In embodiments, complementary DNA (cDNA) encodes a fusion protein as described herein.

Particular embodiments concern the fusion protein consisting of SEQ ID NO: 19, SEQ ID NO: 20, or pharmaceutical compositions of one of these fusion proteins for use as an appetite suppressant, for treatment of obesity, for prevention of obesity, or for treatment of other conditions associated with excess weight in cats.

As described herein, the fusion protein according to any embodiments or combinations of embodiments described herein can be used in the manufacture of a medicament for use as an appetite suppressant, for treatment of obesity, for prevention of obesity, or for treatment of other conditions associated with excess weight in cats.

### BRIEF DESCRIPTION OF THE DRAWINGS

FIG. 1 shows a schematic representation of a GLP-1-analog-Fc fusion protein homodimer. The mutated N-glycosylation site shown is a natural glycosylation site on the Fc fragment which is mutated to prevent glycosylation (glycosylation that may occur on the GLP-1-analog polypeptide is not shown).

FIG. 2 illustrates a side-by-side sequence comparison of the feline proglucagon of SEQ ID NO: 1, the feline GLP-1(1-37) of SEQ ID NO: 2 and the feline GLP-1(7-37) of SEQ ID NO: 3. “*” represents complete homology across all sequences at a given sequence position, while “:”, “.” or spaces refer to conservative, moderate, or very different amino acid mutations across the sequences at a given sequence position, respectively.

FIG. 3 illustrates a side-by-side sequence comparison of the feline GLP-1(7-37) of SEQ ID NO: 3 and the DPP-IV resistant GLP-1-analog of SEQ ID NO: 4. “*” represents complete homology across all sequences at a given sequence position, while “:”, “.” or spaces refer to conservative, moderate, or very different amino acid mutations across the sequences at a given sequence position, respectively.

FIG. 4 illustrates a side-by-side sequence comparison of the GLP-1-analogs of SEQ ID NO: 4, SEQ ID NO: 8 and SEQ ID NO: 14. “*” represents complete homology across all sequences at a given sequence position, while “:”, “.” or spaces refer to conservative, moderate, or very different amino acid mutations across the sequences at a given sequence position, respectively.

FIG. 5A and FIG. 5B illustrate a side-by-side sequence comparison of the Fc fragments of SEQ ID NO: 5, SEQ ID NO: 10, SEQ ID NO: 15, and SEQ ID NO: 18. “*” represents complete homology across all sequences at a given sequence position, while: “:”, “.” or spaces refer to conservative, moderate, or very different amino acid mutations across the sequences at a given sequence position, respectively.

FIG. 6A, FIG. 6B, and FIG. 6C illustrate a side-by-side sequence comparison of the GLP-1-analog-Fc fusion proteins of SEQ ID NO: 9, SEQ ID NO: 11, SEQ ID NO: 12, SEQ ID NO: 16, SEQ ID NO: 17 and SEQ ID NO: 19. “*” represents complete homology across all sequences at a given sequence position, while “:”, “.” or spaces refer to conservative, moderate, or very different amino acid mutations across the sequences at a given sequence position, respectively.

FIG. 7A, FIG. 7B and FIG. 7C illustrate a side-by-side sequence comparison of the GLP-1-analog-Fc fusion proteins of SEQ ID NO: 9, SEQ ID NO: 11, SEQ ID NO: 12, SEQ ID NO: 16, SEQ ID NO: 17 and SEQ ID NO: 20. “*” represents complete homology across all sequences at a given sequence position, while “:”, “.” or spaces refer to conservative, moderate, or very different amino acid mutations across the sequences at a given sequence position, respectively.

FIG. 8 illustrates the concentration level (PK) of the GLP-1-analog-Fc fusion protein in serum over time in cats (N=3) treated with a single dose of the GLP-1-analog-Fc fusion protein of SEQ ID NO: 9.

FIG. 9 illustrates the concentration level (PK) of the GLP-1-analog-Fc fusion protein in serum over time in cats (N=3) treated with a single dose of the GLP-1-analog-Fc fusion protein of SEQ ID NO: 19.

FIG. 10 illustrates the concentration level (PK) of the GLP-1-analog-Fc fusion protein in serum over time in cats (N=3) treated with a single dose of the GLP-1-analog-Fc fusion protein of SEQ ID NO: 20.

FIG. 11 illustrates the GLP-1-analog-Fc fusion protein in vitro GLP-1R binding affinity at various concentrations of the GLP-1-analog-Fc fusion proteins of SEQ ID NO: 9, SEQ ID NO: 11, SEQ ID NO: 12, SEQ ID NO: 16, SEQ ID NO: 17, and SEQ ID NO: 19.

FIG. 12 illustrates the GLP-1-analog-Fc fusion protein in vitro GLP-1R binding affinity at various concentrations of the GLP-1-analog-Fc fusion proteins of SEQ ID NO: 9, SEQ ID NO: 11, SEQ ID NO: 12, SEQ ID NO: 16, SEQ ID NO: 17, and SEQ ID NO: 20.

FIG. 13 illustrates the observed body weight, as a percentage of initial measured body weight, of non-obese, growing cats (N=4) treated with weekly injections of the GLP-1-analog-Fc fusion protein of SEQ ID NO: 19 and a control group of cats treated only with injections of vehicle. GLP-1-analog-Fc fusion protein administration led to less food intake, and therefore leaner cats as compared to the vehicle group.

### DETAILED DESCRIPTION

### Equivalents and Definitions

As used herein, the articles “a” and “an” refer to one or more than one, e.g., to at least one of the grammatical objects of the article. The use of the words “a” or “an” when used in conjunction with the term “comprising” herein may mean “one,” but it is also consistent with the meaning of “one or more,” “at least one,” and “one or more than one.” As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

As used herein, “about” and “approximately” generally mean an acceptable degree of error for the quantity measured given the nature or precision of the measurements.

As used herein, an amount of a molecule, compound, conjugate, or substance effective to treat a disorder (e.g., a disorder described herein), “therapeutically effective amount,” or “effective amount” generally means an amount of the molecule, compound, conjugate, or substance which is effective, upon single or multiple dose administration(s) to a patient, in treating a patient, or in curing, alleviating (e.g., alleviating associated symptoms such as pain or shrinking the size of a tumor), relieving or improving a patient with a condition or disorder (e.g., a disorder described herein) beyond that expected in the absence of such treatment.

As used herein, the term “analog” generally means a compound or conjugate (e.g., a compound or conjugate as described herein) having a chemical structure similar to that of another compound or conjugate but differing from it in at least one aspect.

As used herein, the term “GLP-1-analog” generally means a protein comprising a peptide derived from or consisting of all or a portion of a GLP-1(7-37) protein. For example, a GLP-1-analog may be a native (wild-type) GLP-1(7-37) protein with no changes or mutations, a native GLP-1(7-37) protein comprising an additional peptide sequence combined with the native GLP-1(7-37) protein, or a native GLP-1(7-37) protein with none, one or more than one amino acid deletions, mutations, or additions from the native GLP-1(7-37) protein. In examples, a GLP-1-analog may comprise a portion (e.g., a fragment) or truncated section of a native GLP-1(7-37) protein, and the truncated section of the native GLP-1(7-37) protein may have none, one or more than one amino acid deletions, mutations, or additions. In examples, a GLP-1-analog may comprise a portion of an artificial sequence combined with all or a portion of a native GLP-1(7-37) protein, which may have none, one, or more than one amino acid deletions, mutations, or additions. In examples the GLP-1-analog may comprise a feline GLP-1(7-37) protein as given in SEQ ID NO: 3. In examples, a GLP-1-analog may comprise a mutated feline GLP-1(7-37) protein as given in SEQ ID NO: 4. In examples, a GLP-1-analog may additionally comprise a leader sequence, for example a leader sequence comprising a feline thrombin signal peptide sequence or a mutated feline thrombin signal peptide sequence. In examples, a GLP-1-analog may additionally comprise the feline thrombin signal peptide leader sequence of QHVFLAPQQALSLLQRVRR (SEQ ID NO: 7) or the mutated feline thrombin signal peptide leader sequence of QHVFLAPQQALSLLQRVHD (SEQ ID NO: 13).

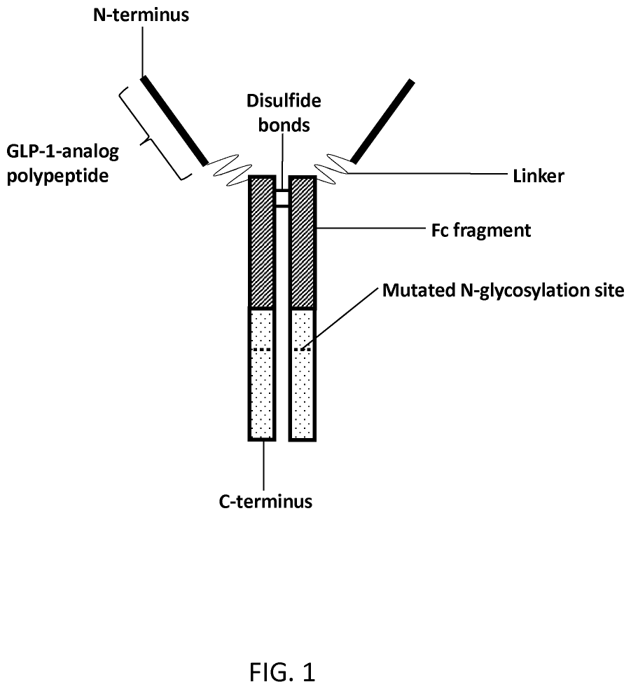

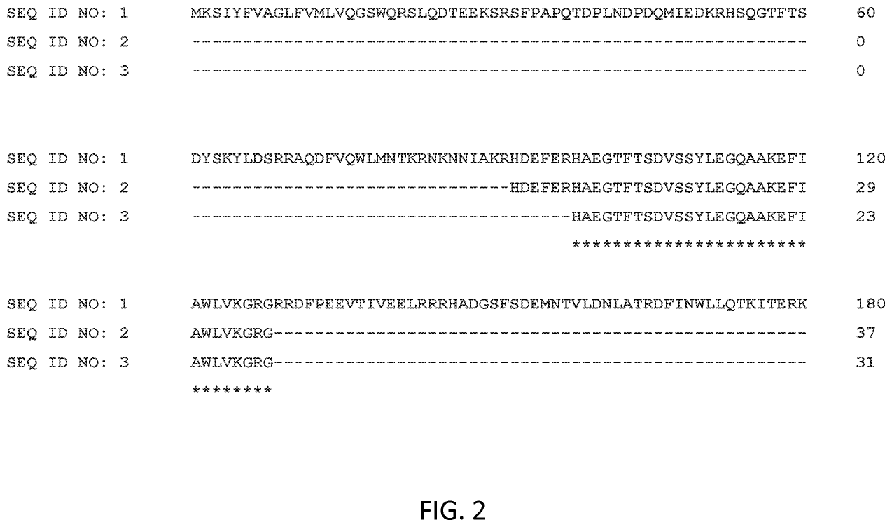

In examples, the GLP-1-analog may be linked to an Fc fragment or analog thereof, as illustrated in FIG. 1. In examples, a GLP-1-analog may or may not have one or more amino acid mutations as compared to a non-mutated GLP-1(7-37) (e.g., a GLP-1(7-37) sequence that is homologous to the feline GLP-1(7-37) of SEQ ID NO: 3).

As used herein, the term “native” generally means a GLP-1-analog or an Fc fragment used in a GLP-1-analog-Fc fusion protein where the species of the GLP-1-analog and/or Fc fragment and the species that the GLP-1-analog-Fc fusion protein is targeted towards are the same. For example, a feline native (wild-type) GLP-1(7-37) protein with no changes or mutations utilized in a GLP-1-analog-Fc fusion protein for use in a feline would be considered a “native” GLP-1-analog. Similarly in a further example, a feline Fc fragment (e.g., a feline native (wild-type) Fc fragment with no changes or mutations) utilized in a GLP-1-analog-Fc fusion protein for use in a feline would be considered a “native” Fc fragment.

As used herein, the term “satiety” generally means the feeling or state of being fully satisfied, after eating. Satiety occurs when the body signals the brain that it has had enough food, reducing the desire to eat more. This sensation helps regulate food intake and maintain energy balance.

As used herein the terms “obese” and “obesity” generally mean a medical condition characterized by excessive body fat that can negatively impact health.

As used herein, the term “dimer” generally means a protein or a fusion protein comprising two polypeptides linked covalently. In embodiments, two identical polypeptides are linked covalently (e.g., via disulfide bonds) forming a “homodimer” (diagrammatically represented in FIG. 1, which is an illustration of a GLP-1-analog-Fc fusion protein homodimer). Referring to FIG. 1 in more detail, the GLP-1-analog polypeptide is connected via a linker to an Fc fragment. Disulfide bonds (the total number of disulfide bonds in actuality may be greater or less than the number shown in FIG. 1) create a homodimer from two identical Fc fusion proteins through linkage of covalent disulfide between individual polypeptide chains. The novel GLP-1-analog-Fc fusion protein homodimer may be encoded by a single nucleic acid molecule, wherein the homodimer is made recombinantly inside a cell by first forming novel GLP-1-analog-Fc fusion protein monomers and by then assembling two identical novel GLP-1-analog-Fc fusion protein monomers into the homodimer upon further processing inside the cell.

As used herein, the terms “multimer,” “multimeric,” or “multimeric state” generally mean non-covalent, associated forms of Fc fusion protein dimers that may be in equilibrium with Fc fusion protein dimers or may act as permanently aggregated versions of Fc fusion protein dimers (e.g., dimers of Fc fusion protein homodimers, trimers of Fc fusion protein homodimers, tetramers of Fc fusion protein homodimers, or higher order aggregates containing five or more Fc fusion protein homodimers). It may be expected that multimeric forms of Fc fusion proteins may have different physical, stability, or pharmacologic activities from that of fusion protein homodimers.

As used herein, a “GLP-1-analog-Fc fusion protein” and a “novel GLP-1-analog-Fc fusion protein” (which terms may be interchangeably used) generally mean an immunoglobin Fc domain that is linked to a GLP-1-analog via a linker peptide, which is useful in binding the GLP-1 receptor in vivo.

As used herein, the term “activity,” “biological activity,” “potency,” “bioactive potency,” or “biological potency” generally means the extent to which an Fc fusion protein binds to or activates a cell receptor and/or exerts the production or reduction of native or foreign substances. As used herein, “in vitro activity” or “receptor activity” generally means the affinity with which an Fc fusion protein binds to a cell receptor and is typically measured by the concentration of an Fc fusion protein that causes the Fc fusion protein to reach half of its maximum binding (i.e., EC50 value). Low EC50 values indicate stronger binding of a GLP-1-analog or GLP-1-analog-Fc fusion protein to a GLP-1R, while high EC50 values indicate weaker binding to a GLP-1R.

As used herein, the term “biosynthesis,” “recombinant synthesis,” or “recombinantly made” generally means the process by which an Fc fusion protein is expressed within a host cell by transfecting the cell with a nucleic acid molecule (e.g., vector) encoding the Fc fusion protein (e.g., where the entire Fc fusion protein is encoded by a single nucleic acid molecule). Exemplary host cells include mammalian cells, e.g., CHO cells or HEK293 cells. The cells can be cultured using standard methods in the art and the expressed Fc fusion protein may be harvested and purified from the cell culture using standard methods in the art.

As used herein, the term “cell surface receptor” generally means a molecule such as a protein, generally found on the external surface of the membrane of a cell and which interacts with soluble molecules, e.g., molecules that circulate in the blood supply. In some embodiments, a cell surface receptor may include a cell receptor (e.g., a GLP-1R) or an Fc receptor which binds to an Fc fragment or the Fc region of an antibody (e.g., an Fc(gamma) receptor, for example Fc(gamma) receptor I (or the equivalent feline or feline Fc(gamma) receptor), or an Fc neonatal receptor, for example FcRn). As used herein, “in vitro activity” or “Fc(gamma) receptor activity” or “Fc(gamma) receptor binding” or “FcRn receptor activity” or “FcRn binding” generally means the affinity with which an Fc fusion protein binds to the Fc receptor (e.g., Fc(gamma) receptor or FcRn receptor) and is typically measured by the concentration of an Fc fusion protein that causes the Fc fusion protein to reach half of its maximum binding (i.e., EC50 value) as measured on an assay (e.g., an enzyme-linked immunosorbent assay (ELISA) assay) using OD 450 nm values (e.g., OD450) as measured on a microplate reader.

As used herein, the term “immunogenic” or “immunogenicity” generally means the capacity for a given molecule or antigen (e.g., an Fc fusion protein of the present invention) to provoke the immune system of a target patient such that after administration (e.g., a single administration or multiple administrations) of the molecule, the patient develops antibodies capable of binding all or specific portions of the molecule (i.e., anti-drug antibodies or ADA). In patients, (e.g., felines) the antibody development may be polyclonal (e.g., a mixture of antibodies capable of binding an Fc fusion protein). As used herein, the terms “neutralizing,” “neutralizing antibodies,” or “neutralizing anti-drug antibodies” generally mean the capacity for antibodies developed against an Fc fusion protein to cross-react, bind and interfere with all or a portion of the Fc fusion protein's biological activity in the target patient. For example, in the case of a novel GLP-1-analog-Fc fusion protein molecule (or a pharmaceutical composition thereof) administered to cats, immunogenicity generally means antibodies that bind to the GLP-1-analog-Fc fusion protein and inhibit the binding between the GLP-1-analog Fc fusion protein and the GLP-1 receptor in a cat.

As used herein, the term “therapeutic composition” or “prophylactic composition” generally means a pharmaceutical composition or mixture of substances comprising an therapeutic or prophylactic molecule that is suitable for administering to a patient. For example, a therapeutic or prophylactic composition may comprise a GLP-1-analog-Fc fusion protein and a sterile aqueous solution or another carrier.

As used herein, the term “monomer” generally means a protein or a fusion protein comprising a single polypeptide. In embodiments, the “monomer” is a protein or a fusion protein, e.g., a single polypeptide, comprising a GLP-1-analog polypeptide and an Fc fragment polypeptide, wherein the GLP-1-analog and the Fc fragment polypeptides are joined by peptide bonds via a linker to form the single polypeptide. In embodiments, the monomer is encoded by a single nucleic acid molecule.

As used herein and as illustrated in FIG. 1, “N-terminus” refers to the start of a protein or polypeptide that is initiated by an amino acid containing a free amine group that is the alpha-amino group of the amino acid (e.g., the free amino that is covalently linked to one carbon atom that is located adjacent to a second carbon atom, wherein the second carbon atom is part of the carbonyl group of the amino acid). As used herein and as illustrated in FIG. 1, “C-terminus” refers to the end of a protein or polypeptide that is terminated by an amino acid containing a carboxylic acid group, wherein the carbon atom of the carboxylic acid group is located adjacent to the alpha-amino group of the amino acid.

As used herein, the term “carrier” is used herein to include diluents, excipients, vehicles, and the like, in which the Fc fusion protein(s) may be dispersed, emulsified, or encapsulated for administration. Suitable carriers will be pharmaceutically acceptable. As used herein, the term “pharmaceutically acceptable” generally means not biologically or otherwise undesirable, in that it can be administered to a patient without excessive toxicity, irritation, or allergic response, and does not cause unacceptable biological effects or interact in a deleterious manner with any of the other components of the composition in which it is contained. A pharmaceutically acceptable carrier would naturally be selected to minimize any degradation of the compound or other agents and to minimize any adverse side effects in the patient, as would be well known to one of skill in the art. Pharmaceutically acceptable ingredients include those acceptable for veterinary use as well as human pharmaceutical use and will depend on the route of administration. Any carrier compatible with the excipient(s) and the Fc fusion protein(s) can be used.

As used herein, a “T-cell epitope” is a specific fragment of an antigen that is recognized and bound by T-cell receptors (TCRs) on the surface of T-cells. These epitopes are crucial for the activation of T-cells and the initiation of an adaptive immune response.

As used herein, “pharmacodynamics” or “PD” generally means the biological effects of an Fc fusion protein in a patient. As an example, herein, the PD of a novel GLP-1-analog-Fc fusion protein generally means the ability of the GLP-1-analog-Fc fusion protein to reduce weight levels over time in a patient after the administration of the novel GLP-1-analog-Fc fusion protein.

As used herein, “pharmacokinetics” or “PK” generally means the characteristic interactions of an Fc fusion protein and the body of the patient in terms of its absorption, distribution, metabolism, and excretion. As an example, herein, the PK generally means the concentration of a novel GLP-1-analog-Fc fusion protein in the blood or serum of a patient at a given time after the administration of the novel GLP-1-analog-Fc fusion protein. As used herein, “pharmacokinetic half-life” or, more generically “half-life”, generally means the time taken for the concentration of Fc fusion protein in the blood or serum of a patient to reach half of its original value as calculated from a first order exponential decay model for drug elimination. Fc fusion proteins with greater “half-life” values demonstrate greater duration of action in the target patient.

As used herein, “structural half-life” generally means the time taken for the concentration of a given GLP-1 peptide, GLP-1-analog peptide or GLP-1-analog peptide portion of a GLP-1-Fc fusion protein amino acid sequence to remain intact and substantially unchanged in the blood or serum of a patient to reach half of its original value as calculated from a first order exponential decay model.

As used herein, “duration of activity” generally means the time during which an Fc fusion protein is detectable in the blood serum of a subject relative to a pre-dose level. The duration of activity of an Fc fusion protein in vivo generally depends on the half-life of the Fc fusion protein in vivo.

The terms “sequence identity,” “sequence homology,” “homology,” or “identical” in amino acid or nucleotide sequences as used herein describes that the same nucleotides or amino acid residues are found within the variant and reference sequences when a specified, contiguous segment of the nucleotide sequence or amino acid sequence of the variant is aligned and compared to the nucleotide sequence or amino acid sequence of the reference sequence. Methods for sequence alignment and for determining identity between sequences are known in the art, including the use of Clustal Omega, which organizes, aligns, and compares sequences for similarity, wherein the software highlights each sequence position and compares across all sequences at that position and assigns one of the following scores: an “*” (asterisk) for sequence positions which have a single, fully conserved residue, a “:” (colon) indicates conservation between groups of strongly similar properties with scoring greater than 0.5 in the Gonnet PAM 250 matrix, and a “.” (period) indicates conservation between groups of weakly similar properties with scoring less than or equal to 0.5 in the Gonnet PAM 250 matrix, a “-” (dash) indicates a sequence gap, meaning that no local homology exists within a particular set of comparisons within a certain range of the sequences, and an empty space “ ” indicates little or no sequence homology for that particular position across the compared sequences.

With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. In some embodiments, the contiguous segment used for comparison to the reference nucleotide sequence or reference amino acid sequence will comprise at least 6, 10, 15, or 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are known in the art.

As used herein, the term “homology” is used to compare two or more proteins by locating common structural characteristics and common spatial distribution of, for instance, beta strands, helices, and folds. Accordingly, homologous protein structures are defined by spatial analyses. Measuring structural homology involves computing the geometric-topological features of a space. One approach used to generate and analyze three-dimensional (3D) protein structures is homology modeling (also called comparative modeling or knowledge-based modeling) which works by finding similar sequences on the basis of the fact that 3D similarity reflects 2D similarity. Homologous structures do not imply sequence similarity as a necessary condition.

As used herein, the determination of percent identity or “homology” between two sequences is accomplished using a mathematical algorithm. For example, the percent identity of an amino acid sequence is determined using the Smith-Waterman homology search algorithm using an affine 6 gap search with a gap open penalty of 12 and a gap extension penalty of 2, BLOSUM matrix 62. In embodiments, the percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic.

As used herein, the terms “subject” or “target subject” or “patient” or “patient subject” or “pets” or “companion animals” are intended to include mammals, including felines. Exemplary feline subjects or patients include cats having a condition, disease, or a disorder, e.g., obesity or other diseases associated with excess weight described herein, or normal subjects.

As used herein, the term “titer” or “yield” generally means the amount of a fusion protein product (e.g., an Fc fusion protein described herein) resulting from the biosynthesis (e.g., in a mammalian cell, e.g., in a HEK293 cell or CHO cell) per volume of the cell culture. The amount of product may be determined at any step of the production process (e.g., before or after purification), but the yield or titer is always stated per volume of the original cell culture. As used herein, the term “product yield” or “total protein yield” generally means the total amount of Fc fusion protein expressed by cells and purified via at least one affinity chromatography step (e.g., Protein A or Protein G) and includes monomers of Fc fusion protein, homodimers of Fc fusion protein, and higher-order molecular aggregates of homodimers of Fc fusion protein. As used herein, the term “percent homodimer” or “% homodimer” generally means the proportion of a fusion protein product (e.g., an Fc fusion protein described herein) that is the desired homodimer.

As used herein, the terms “treat” or “treating” or “treatment” of a patient having a condition, disease, or a disorder generally mean an intervention performed with the intention of mitigating or preventing the symptoms (in particular, a treatment of obesity or other diseases associated with excess weight) associated with the condition or disease, and/or reducing the duration of the symptoms associated with the condition or disease. Accordingly, “treatment” generally means both therapeutic treatment and prophylactic or preventative measures. Improvement after treatment may be manifested as a decrease or elimination of such symptoms, e.g., by a decrease in body weight, and/or by a prevention of excessive weight gain and the onset of obesity. Treating a patient having a condition or disease may include subjecting the patient with the condition or disease to a treatment regimen, for example the administration of a fusion protein such as a GLP-1-analog-Fc fusion protein described herein, or a pharmaceutical composition of a fusion protein such as a GLP-1-analog-Fc fusion protein described herein, such that the obesity or other diseases associated with excess weight are reduced, alleviated, relieved, altered, remedied, ameliorated, or improved, such that excessive weight gain is prevented during the course of the treatment. Treating includes administering an amount of a fusion protein such as a GLP-1-analog-Fc fusion protein described herein, or a pharmaceutical composition of a fusion protein such as a GLP-1-analog-Fc fusion protein described herein that is effective to reduce, alleviate, relieve, alter, remedy, ameliorate, and/or improve, the health of a patient with obesity or other diseases associated with excess weight.

As used herein, the phrase “effective amount” or “therapeutically effective amount” generally means a therapeutic or prophylactic amount of the GLP-1-analog-Fc fusion protein of the present disclosure or pharmaceutical composition thereof, that elicit the desired therapeutic or prophylactic effect on obesity or the prevention of weight gain or other diseases associated with excess weight as evidenced by the alleviation of some or all of such symptoms of the condition or disease, when administered in accordance with the desired treatment regimen. In examples, where the desired therapeutic or prophylactic effect or response is to alleviate symptoms of a condition or disease, an amount of a GLP-1-analog-Fc fusion protein of the present disclosure or pharmaceutical composition thereof may be considered therapeutically effective if symptoms and/or effects of the condition or disease are observably reduced in the patient after the treatment regime. The therapeutically effective dosage of a GLP-1-analog-Fc fusion protein may vary depending on the size and species of the patient, and/or according to the mode of administration.

As used herein, when referring to an amino acid in some portion of an amino acid sequence, for example a GLP-1-analog amino acid sequence, a cited amino acid position is referenced as the position of the amino acid counting from the beginning of the amino acid sequence itself. For example, consider the feline GLP-1(7-37) amino acid sequence of SEQ ID NO: 3.

(SEQ ID NO: 3)
HAEGTFTSDVSSYLEGQAAKEFIAWLVKGRG.

A mutation of the first alanine amino acid of this sequence would be described as a mutation of the 2^{nd} amino acid of the sequence. For example, if the first alanine amino acid in SEQ ID NO: 3 were mutated to glycine, this could be referred to as an A2G mutation of SEQ ID NO: 3.

As used herein, “use as an appetite suppressant, for treatment of obesity, for prevention of obesity, or for treatment of other conditions associated with excess weight in cats” generally means the treatment of any condition which can be treated by the application of GLP-1(7-37).

### GLP-1 Treatments for Obesity

GLP-1 treatments promote weight loss by reducing feeding frequency and meal size. GLP-1 reduces the desire to eat and increases feelings of fullness (satiety). In addition, GLP-1 release leads to a decrease in gastric emptying, meaning food stays in the stomach longer. This delay contributes to prolonged feelings of fullness and reduced hunger. GLP-1 can also influence other hormones related to hunger and satiety, such as glucagon, further promoting a sense of fullness and reducing food intake. GLP-1 has many uses that could be used to treat obesity by increasing satiety (feeling of fullness) which in turn reduces feeding frequency and meal size. GLP-1 also promotes weight loss through decreasing the rate of gastric emptying. GLP-1 also inhibits glucagon release. In contrast, the glucagon hormone has an opposing biological activity and plays a major role in increasing glucose levels in the blood.

GLP-1-analogs that have been engineered to have an increased pharmacokinetic half-life are used in human medicine to treat obesity. These GLP-1-analogs can increase the physiological effect of satiety (feeling of fullness) which suppresses appetite and energy intake in both normal weight, overweight and obese individuals. As a result, GLP-1-analogs with an increased pharmacokinetic half-life have become important therapeutics for weight loss in humans.

Long acting GLP-1 formulations would have dramatic effects on the survival and the quality of life of cats. Further, the role played by GLP-1 in increasing satiety makes GLP-1 formulations an attractive target for treating weight gain and obesity in cats.

The use of GLP-1 as an appetite suppressant by inducing the effect of satiety makes GLP-1 a potential prevention of, and treatment of obesity and other associated conditions caused by being overweight. A treatment for use as an appetite suppressant, for treatment of obesity, for prevention of obesity, or for treatment of other conditions associated with excess weight in cats, that preferably requires dosing by twice-weekly, once-weekly, or biweekly injections in order to maximize owner compliance, is expected to lead to fewer complications associated with pet obesity, and better outcomes for the pets.

GLP-1-analogs, for example Semaglutide (Ozempic®) and other drugs that increase the pharmacokinetic half-life of GLP-1, are used worldwide in human medicine to treat obesity. Semaglutide has an extended half-life mechanism that relies on a fatty acid chain-containing GLP-1-analog that binds to circulating albumin in vivo, thereby preventing renal clearance. These drugs can increase the feeling of satiety which in turn reduces feeding frequency and meal size. GLP-1-analogs also promote weight loss through decreasing the rate of gastric emptying, which contributes to prolonged feelings of fullness and reduced hunger. GLP-1 is additionally known to affect regions of the brain involved with appetite and reward, such as the amygdala hypothalamus, and insula.

GLP-1-analog-Fc fusion proteins have been developed for treating humans that make use of a human Fc region to prolong the GLP-1 action in vivo. These human medicants include Dulaglutide (Trulicity®) developed by Eli Lilly (Indianapolis, United States) which was approved by the FDA in 2014. This is a fusion protein of a mutated GLP-1(7-37) linked (via a linker) to a modified human IgG4 Fc fragment. The GLP-1-analog used in Dulaglutide includes 3 mutations to the native GLP-1(7-37) (SEQ ID NO: 3) to create the GLP-1-analog of SEQ ID NO: 4. The GLP-1-analog of SEQ ID NO: 4 is shown below.

(SEQ ID NO: 4)
HGEGTFTSDVSSYLEEQAAKEFIAWLVKGGG.

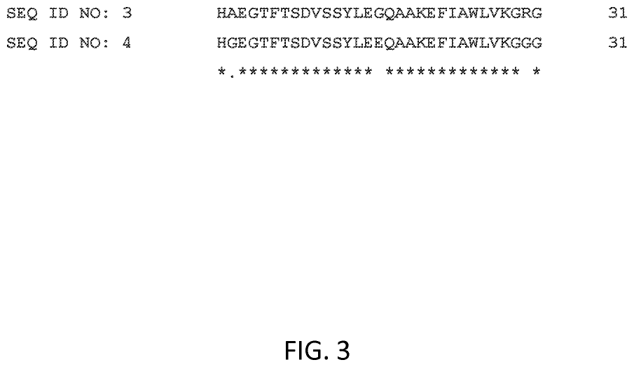

A Clustal Omega comparison of the native GLP-1(7-37) SEQ ID NO: 3 vs the GLP-1-analog of SEQ ID NO: 4 is shown in FIG. 3.

As shown in FIG. 3, the GLP-1-analog of SEQ ID NO: 4 includes three mutations: an alanine mutated to glycine at position 2 (A2G), a glycine mutated to glutamic acid at position 16 (G16E), and an arginine mutated to glycine at position 30 (R30G). The mutation of alanine to glycine at position 2 (A2G) of GLP-1(7-37) of SEQ ID NO: 3 renders the peptide resistant to DPP-IV (Gilor. C. et al. (2016). New approaches to feline diabetes mellitus, Glucagon-like peptide-I analogs. Journal of Feline Medicine and Surgery 18, 733-743). The mutation of the glycine to glutamic acid at position 16 (G16E) increases solubility. Substituting an arginine residue for glycine at position 30 (R30G) reduces immunogenicity by removing a potential T-cell epitope.

In addition, in Dulaglutide, two GLP-1-analog molecules are covalently linked to an Fc fragment as a homodimer, wherein the Fc fragment is of the human IgG4 type which is glycosylated. This makes a large-size molecule that slows absorption from the subcutaneous injection site and prolongs the pharmacokinetic half-life considerably by reducing renal clearance. Dulaglutide has a pharmacokinetic half-life of 5 days in people. However, GLP-1-analog-Fc fusion proteins such as Dulaglutide with a human Fc, when used in cats, are expected to develop antibodies in cats against the foreign human Fc sequence after several administrations, and the resulting antibodies will neutralize dulaglutide action in cats.

### Novel GLP-1-Analog-Fc Fusion Proteins

As disclosed herein, proposed ultra-long acting GLP-1 treatments for veterinary clinical use comprise a GLP-1-analog-Fc fusion protein making use of an Fc fragment to prolong their action in vivo. A GLP-1-analog-Fc fusion protein suitable for an ultra-long acting treatment for use as an appetite suppressant, for treatment of obesity, for prevention of obesity, or for treatment of other conditions associated with excess weight in cats, should meet various design goals. A GLP-1-analog-Fc fusion protein configuration suitable for an ultra-long acting treatment in cats should be manufacturable in mammalian cells, for example human embryonic kidney (HEK, e.g., HEK293) cells or Chinese hamster ovary (CHO) cells, with an acceptable titer of the desired homodimer product (e.g., greater than 200 mg/L manufacturing titer from transiently transfected CHO cells, greater than 250 mg/L from transiently transfected CHO cells, greater than 300 mg/L from transiently transfected CHO cells, etc.). Only candidates with a manufacturing titer of greater than 300 mg/L from transiently transfected CHO cells are considered useful in the present invention, because experience has demonstrated that manufacturing titers less than this level will not likely result in commercial production manufacturing titers in stably transfected CHO cells that meet the stringently low manufacturing cost requirements for veterinary products. Additionally, GLP-1-analog-Fc fusion proteins should be manufacturable without a high level of aggregates produced, as assessed by the homodimer percentage (% homodimer) of the manufactured compound. The % homodimer of a candidate GLP-1-analog-Fc fusion protein should be greater than or equal to 85%.

Previous work with insulin-Fc fusion proteins, such as is described in WO2020006529A1, has demonstrated that the choices of the protein sequence, the linker sequence, and the composition of the Fc domain can all potentially influence protein yields, purity, and pharmacodynamics. The application WO2020006529A1 describes a combination of a different insulin analog, linker and Fc domain that yield pharmacodynamically active insulin-Fc fusion proteins. The application WO2020006529A1 describes combinations of insulin analogs, linkers and Fc domains that have poor manufacturability or homogeneity. In the case of the application WO2020006529A1, the specific aim was to produce an insulin-Fc fusion protein that did not generate antibodies.

In choosing the GLP-1-analog for the novel GLP-1-analog-Fc fusion protein it is conceivable that one could choose a GLP-1-analog that includes some portion of a wild-type GLP-1 protein sequence. For example, the GLP-1 for a novel GLP-1-analog-Fc fusion protein may comprise all or a portion of the mutated feline GLP-1-analog protein sequence of SEQ ID NO: 4, or may comprise all or a portion of the native feline GLP-1(1-37) protein sequence of SEQ ID NO: 2 or may comprise all or a portion of the native feline GLP-1(7-37) protein sequence of SEQ ID NO: 3. In examples, the GLP-1-analog may comprise all or a portion of a native GLP-1 protein sequence with additional amino acids or polypeptides, (collectively then referred to as the GLP-1-analog). In examples, one or more amino acids in the GLP-1-analog of the novel GLP-1-analog-Fc fusion protein may be deleted or mutated from their native state.

It is expected that different GLP-1-analog-Fc fusion protein designs will result in different protein yields (see for example, Beygmoradi et al., (2023). Recombinant protein expression: Challenges in production and folding related matters, International Journal of Biological Macromolecules, Volume 233, 123407, ISSN 0141-8130, doi.org 10.10161j.ijbiomac.2023.123407, and Massimo Stefani, (2004). Protein misfolding and aggregation: new examples in medicine and biology of the dark side of the protein world, Biochimica et Biophysica Acta (BBA)—Molecular Basis of Disease, Volume 1739, Issue 1, Pages 5-25, ISSN 0925-4439.)

For example, larger or shorter GLP-1(7-37) sequences in the GLP-1-analog, when used to produce the GLP-1-analog-Fc fusion protein, are expected to result in different protein yields. The resulting protein yield when the selected GLP-1-analog is attached to an Fc fragment can be experimentally determined. The choice of the Fc fragment and the portion of the Fc fragment hinge region that is linked to the selected GLP-1-analog is expected to impact the manufacturability of the GLP-1-analog-Fc fusion protein.

The novel GLP-1-analog-Fc fusion protein may comprise a peptide linker. In examples, the protein comprising the GLP-1-analog is linked to the N-terminal side of the Fc fragment. In examples, the novel GLP-1-analog-Fc fusion protein comprises domains in the following orientation from N- to C-termini: (N-terminus)-therapeutic protein-peptide linker-Fc fragment-(C-terminus) (e.g., (N-terminus) GLP-1-analog-peptide linker-Fc fragment-(C-terminus)).

In addition, the GLP-1-analog-Fc fusion protein must bind the GLP-1 receptor with an appreciable affinity (e.g., OD450 of greater than or equal to 0.25 at a GLP-1-analog-Fc fusion protein concentration of 2000 ng/mL) as measured in a GLP-1 receptor binding assay. Based on experience, only fusion proteins exhibiting this level of GLP-1 receptor binding activity are deemed likely to exhibit the requisite bioactivity in the target species. The GLP-1-analog-Fc fusion protein must also demonstrate sustained bioactivity in vivo (e.g., demonstrate reduced food intake over periods of 3 days, 4 days, 5 days, 6 days, 7 days, or longer) or sustained pharmacokinetics in vivo (e.g., measurable serum pharmacokinetics over periods of 3 days, 4 days, 5 days, 6 days, 7 days, or longer) to justify less frequent dosing. To achieve this, the GLP-1-analog-Fc fusion protein cannot bind the GLP-1 receptor too strongly as it will be cleared from the body too quickly via the GLP-1 receptor through receptor-mediated endocytosis. Based on experience, only GLP-1-analog-Fc fusion proteins exhibiting an EC50 greater than or equal to 250 ng/mL as measured in a GLP-1 receptor binding assay will demonstrate sufficient system residence time in the target animal, provided that they demonstrate at least some minimal binding as measured in a GLP-1 receptor binding assay (e.g., the molecule has an appreciable OD450 value at a GLP-1-analog-Fc fusion protein concentration of 2000 ng/mL).

There are three feline IgG Fc isotypes: IgG1a, IgG1b, and IgG2. Replacing a human Fc with a feline IgG Fc isotype would help to prevent unwanted immunogenicity in cats and prevent the creation of neutralizing anti-drug antibodies against the foreign human Fc sequence. IgG subclasses in felines have different molecular properties due to variations in their structure which can affect their function (e.g., receptor binding and/or complement activation) and other properties such as manufacturability.

A first design goal is to create a GLP-1-analog-Fc fusion protein with an acceptable protein yield after production in transiently transfected CHO cells and protein A purification. A design goal of over 300 mg/L was determined to meet the stringent cost requirements of this therapeutic as a treatment in the animal health space.

A second design goal is to create a GLP-1-analog-Fc fusion protein with an appropriate GLP-1 receptor binding affinity to enable a long in vivo pharmacokinetic half-life while still allowing for some engagement and binding between the GLP-1-analog of the GLP-1-analog-Fc fusion protein and the GLP-1 receptor in vivo. If the receptor binding is too high then the in vivo clearance rate of the GLP-1-analog-Fc fusion protein will likely be too short, while if the GLP-1 receptor binding is too low, then the GLP-1-analog-Fc fusion protein may not be able to activate the GLP-1 receptor to obtain the desired effect in vivo (e.g., increased sensation of satiety, lower food intake, weight loss and treatment of obesity). There is a minimum threshold binding level that the GLP-1-analog portion of the fusion protein must exceed, while at the same time not having too high of an affinity for the GLP-1 receptor. The affinity between the GLP-1-analog-Fc fusion protein and the GLP-1 receptor can be measured in a GLP-1R ELISA. Therefore, using this GLP-1R ELISA the following additional GLP-1 receptor binding affinity assay design goals were created: (i) at a GLP-1-analog-Fc fusion protein concentration of 2000 ng/mL, the GLP-1-analog-Fc fusion protein should have an OD450 greater than or equal to 0.25 to ensure that there is some level of binding and (ii) the EC50 calculated from the GLP-1 receptor binding affinity assay should be greater than or equal to 250 ng/mL to ensure that binding to the GLP-1 receptor is not too high to minimize unwanted clearance of the GLP-1-analog-Fc fusion protein in vivo.

A third design goal is to create a GLP-1-analog-Fc fusion protein with a long acting duration in vivo, i.e., to create a GLP-1-analog-Fc fusion protein that has a pharmacokinetic half-life of over 72 hours in vivo in order to be used as a potential twice weekly injection, which would be a significant improvement on the current daily or twice daily treatment options.

A fourth design goal is to create a GLP-1-analog-Fc fusion protein with low immunogenicity (no anti-drug antibodies produced) as this would also result in the fusion protein being removed from the body quickly or being neutralized in a manner that would not enable it to bind to the GLP-1 receptor, both of which would result in the fusion protein being rendered inefficacious.

As a first attempt in creating a novel GLP-1-analog-Fc fusion protein for use in cats, a feline GLP-1-Fc sequence from the prior art was selected. As explained in WO2022046809A1, this prior art feline GLP-1-Fc sequence was developed for use as a viral vector delivered using Adena-Associated-Virus (AAV) technology to promote sustained expression of the GLP-1 receptor agonist in felines in vivo. The sequence utilized a DPP-IV resistant GLP-1-analog which was expected to prevent loss of potency due to in vivo enzymatic clipping of the GLP-1-analog. The DPP-IV resistant GLP-1-analog is shown as SEQ ID NO: 4 below.

(SEQ ID NO: 4)
HGEGTFTSDVSSYLEEQAAKEFIAWLVKGGG.

As shown in FIG. 3, the GLP-1-analog of SEQ ID NO: 4 includes three mutations from the native feline GLP-1(7-37) of SEQ ID NO: 3 in an attempt to improve the structural half-life against enzymatic degradation. The alanine at position 2 is mutated to glycine (A2G) to render the GLP-1 portion resistant to DPP-IV (Gilor. C. et al. (2016). New approaches to feline diabetes mellitus, Glucagon-like peptide-1 analogs. Journal of Feline Medicine and Surgery 18, 733-743). The glycine at position 16 is mutated to glutamic acid (G16E) to increase solubility. The arginine at position 30 is mutated to glycine (R30G) to reduce immunogenicity by removing a potential T-cell epitope. In addition, a feline thrombin signal peptide as a leader sequence was added and was expected to allow for thrombin enzymatic cleavage in vivo to expose the GLP-1 portion of the molecule, which was used in the prior art sequence as it appeared to confer advantages to the expression of the feline GLP-1-Fc in vivo. The feline thrombin signal peptide leader sequence is shown as SEQ ID NO: 7 below.

(SEQ ID NO: 7)
QHVFLAPQQALSLLQRVRR.

These two sequences combined comprise the GLP-1-analog used in the GLP-1-analog-Fc fusion protein. The GLP-1-analog is shown as SEQ ID NO: 8 below.

(SEQ ID NO: 8)
QHVFLAPQQALSLLQRVRRHGEGTFTSDVSSYLEEQAAKEFIAWLVKG
GG.

The GLP-1-analog of SEQ ID NO: 8 was linked to a feline IgG1a Fc fragment which included a hinge region of the Fc fragment. The feline native IgG1a fragment of SEQ ID NO: 5 including the hinge region is shown below:

(SEQ ID NO: 5)
ARKTDHPPGPKPCDCPKCPPPEMLGGPSIFIFPPKPKDTLSISRTPEVT
CLVVDLGPDDSDVQITWFVDNTQVYTAKTSPREEQFNSTYRVVSVLPIL
HQDWLKGKEFKCKVNSKSLPSPIERTISKAKGQPHEPQVYVLPPAQEEL
SRNKVSVTCLIKSFHPPDIAVEWEITGQPEPENNYRTTPPQLDSDGTYF
VYSKLSVDRSHWQRGNTYTCSVSHEALHSHHTQKSLTQSPG.

The GLP-1-analog of SEQ ID NO: 8 was linked via the linker GGGGSGGGGSGGGGS (SEQ ID NO: 6) to the feline IgG1a Fc fragment of SEQ ID NO: 5. The resultant GLP-1-analog-Fc fusion protein is given below as SEQ ID NO: 9.

(SEQ ID NO: 9)
QHVFLAPQQALSLLQRVRRHGEGTFTSDVSSYLEEQAAKEFIAWLVKGG
GGGGGSGGGGSGGGGSARKTDHPPGPKPCDCPKCPPPEMLGGPSIFIFP
PKPKDTLSISRTPEVTCLVVDLGPDDSDVQITWFVDNTQVYTAKTSPRE
EQFNSTYRVVSVLPILHQDWLKGKEFKCKVNSKSLPSPIERTISKAKGQ
PHEPQVYVLPPAQEELSRNKVSVTCLIKSFHPPDIAVEWEITGQPEPEN
NYRTTPPQLDSDGTYFVYSKLSVDRSHWQRGNTYTCSVSHEALHSHHTQ
KSLTQSPG.

The GLP-1-analog-Fc fusion protein of SEQ ID NO: 9 was manufactured in CHO cells according to Example 1 and was purified according to Example 3. The Fc fusion protein structure of the GLP-1-analog-Fc fusion protein of SEQ ID NO: 9 was confirmed according to Example 4, and sequence identification is performed according to Example 5. The resultant yield of the GLP-1-analog-Fc fusion protein produced was 569 mg/L. The homodimer percentage was determined according to Example 6. The resulting homodimer percentage was 81.6%, which did not meet the design goal of % homodimer greater than or equal to 85%.

The manufacturing yield of the GLP-1-analog-Fc fusion protein of SEQ ID NO: 9 did exceed the target level of 300 mg/L. The affinity between the GLP-1-analog-Fc fusion protein and the GLP-1 receptor was measured in a GLP-1R ELISA according to Example 8. Unexpectedly, this GLP-1-analog-Fc fusion protein showed very strong affinity for the GLP-1R (32 ng/mL EC50), which did not meet the design goal. It was expected that such strong GLP-1R affinity would result in a poorer than expected half-life, likely because the strong affinity would cause the molecule to be cleared predominantly through GLP-1R mediated endocytosis.

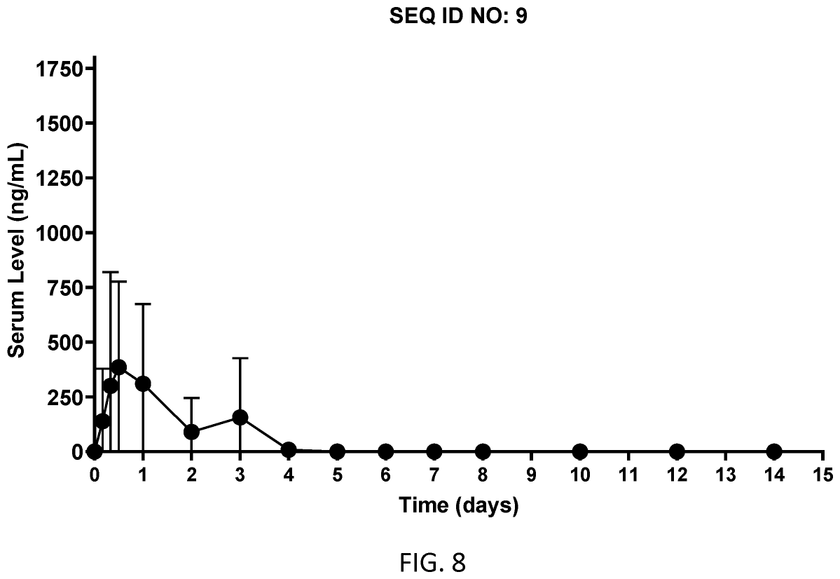

The GLP-1-analog-Fc fusion protein of SEQ ID NO: 9 was administered to N=3 cats according to Example 10 and the pharmacokinetics of the GLP-1-analog-Fc fusion protein of SEQ ID NO: 9 in vivo was measured according to Example 9. The level of the GLP-1-analog-Fc fusion protein measured in serum over time is shown in FIG. 8, demonstrating that the GLP-1-analog-Fc fusion protein of SEQ ID NO: 9 exhibited a pharmacokinetic half-life of just 18 hours (0.75 days), which did not meet the design goal of a pharmacokinetic half-life exceeding 3 days.

IgG subclasses in felines have different molecular properties due to variations in their structure which can affect their function (e.g., receptor binding and/or complement activation) and other properties such as manufacturability. In an effort to understand the impacts of different molecular properties, in a second attempt, the native feline IgG1b was used as the Fc fragment. It was not expected that the change in feline IgG subclass for the Fc fragment would significantly impact the GLP-1R binding affinity. In addition, the upper hinge region of the feline IgG1b Fc fragment was deleted to increase rigidity/sterics in an attempt to lower the GLP-1 receptor binding affinity. The feline native IgG1b fragment of SEQ ID NO: 10 is shown below.

(SEQ ID NO: 10)
DCPKCPPPEMLGGPSIFIFPPKPKDTLSISRTPEVTCLVVDLGPDDSDV
QITWFVDNTQVYTAKTSPREEQFNSTYRVVSVLPILHQDWLKGKEFKCK
VNSKSLPSPIERTISKDKGQPHEPQVYVLPPAQEELSRNKVSVTCLIEG
FYPSDIAVEWEITGQPEPENNYRTTPPQLDSDGTYFLYSRLSVDRSRWQ
RGNTYTCSVSHEALHSHHTQKSLTQSPG.

The GLP-1-analog of SEQ ID NO: 8 was linked via the linker GGGGSGGGGSGGGGS (SEQ ID NO: 6) to the feline IgG1b Fc fragment of SEQ ID NO: 10. The resultant GLP-1-analog-Fc fusion protein is given below as SEQ ID NO: 11.

(SEQ ID NO: 11)
QHVFLAPQQALSLLQRVRRHGEGTFTSDVSSYLEEQAAKEFIAWLVKGG
GGGGGSGGGGSGGGGSDCPKCPPPEMLGGPSIFIFPPKPKDTLSISRTP
EVTCLVVDLGPDDSDVQITWFVDNTQVYTAKTSPREEQFNSTYRVVSVL
PILHQDWLKGKEFKCKVNSKSLPSPIERTISKDKGQPHEPQVYVLPPAQ
EELSRNKVSVTCLIEGFYPSDIAVEWEITGQPEPENNYRTTPPQLDSDG
TYFLYSRLSVDRSRWQRGNTYTCSVSHEALHSHHTQKSLTQSPG.

The GLP-1-analog-Fc fusion protein of SEQ ID NO: 11 was manufactured in CHO cells according to Example 1 and was purified according to Example 3. The Fc fusion protein structure of the GLP-1-analog-Fc fusion protein of SEQ ID NO: 11 was confirmed according to Example 4, and sequence identification is performed according to Example 5. The resultant yield of the GLP-1-analog-Fc fusion protein produced was 252 mg/L. The homodimer percentage was determined according to Example 6. The resulting homodimer percentage was 85.5%, which met the design goal.

The use of a feline IgG1b Fc fragment decreased the manufacturing titer, The manufacturing yield of the GLP-1-analog-Fc fusion protein of SEQ ID NO: 11 did not reach the target level of 300 mg/L.

The affinity between the GLP-1-analog-Fc fusion protein of SEQ ID NO: 11 and the GLP-1 receptor was measured in a GLP-1R ELISA according to Example 8. As expected, the change in feline IgG subclass for the Fc fragment did not appreciably impact the GLP-1R binding affinity, and this GLP-1-analog-Fc fusion protein also showed strong affinity for the GLP-1R (50 ng/mL EC50), which did not meet the design goal, and which is expected to lead to an unacceptably short in vivo pharmacokinetic half-life.

It was considered that the feline thrombin signal peptide leader sequence (SEQ ID NO: 7) in the GLP-1-analog was potentially causing manufacturing problems when combined with an IgG1b Fc fragment.

As a third attempt, a GLP-1-analog without the feline thrombin signal peptide was used. The GLP-1-analog comprised only the DPP-IV resistant GLP-1-analog with mutations including protection from DPP-IV inactivation (A2G), increased solubility (G16E), and reduction of immunogenicity via substituting a glycine residue for arginine (R30G) to remove a potential T-cell epitope. The GLP-1-analog is shown below in SEQ ID NO: 4.

(SEQ ID NO: 4)
HGEGTFTSDVSSYLEEQAAKEFIAWLVKGGG.

The native feline IgG1b was used as the Fc fragment. The upper hinge region of the feline IgG1b Fc fragment was deleted to increase rigidity/sterics in an attempt to lower the GLP-1 receptor binding affinity. The feline native IgG1b fragment of SEQ ID NO: 10 is shown below.

(SEQ ID NO: 10)
DCPKCPPPEMLGGPSIFIFPPKPKDTLSISRTPEVTCLVVDLGPDDSDV
QITWFVDNTQVYTAKTSPREEQFNSTYRVVSVLPILHQDWLKGKEFKCK
VNSKSLPSPIERTISKDKGQPHEPQVYVLPPAQEELSRNKVSVTCLIEG
FYPSDIAVEWEITGQPEPENNYRTTPPQLDSDGTYFLYSRLSVDRSRWQ
RGNTYTCSVSHEALHSHHTQKSLTQSPG.

The GLP-1-analog of SEQ ID NO: 4 was linked via the linker GGGGSGGGGSGGGGS (SEQ ID NO: 6) to the feline IgG1b Fc fragment of SEQ ID NO: 10. The resultant GLP-1-analog-Fc fusion protein is given below as SEQ ID NO: 12.

(SEQ ID NO: 12)
HGEGTFTSDVSSYLEEQAAKEFIAWLVKGGGGGGGSGGGGSGGGGSDCP
KCPPPEMLGGPSIFIFPPKPKDTLSISRTPEVTCLVVDLGPDDSDVQIT
WFVDNTQVYTAKTSPREEQFNSTYRVVSVLPILHQDWLKGKEFKCKVNS
KSLPSPIERTISKDKGQPHEPQVYVLPPAQEELSRNKVSVTCLIEGFYP
SDIAVEWEITGQPEPENNYRTTPPQLDSDGTYFLYSRLSVDRSRWQRGN
TYTCSVSHEALHSHHTQKSLTQSPG.

The GLP-1-analog-Fc fusion protein of SEQ ID NO: 12 was manufactured in CHO cells according to Example 1 and was purified according to Example 3. The Fc fusion protein structure of the GLP-1-analog-Fc fusion protein of SEQ ID NO: 12 was confirmed according to Example 4, and sequence identification is performed according to Example 5. The resultant yield of the GLP-1-analog-Fc fusion protein produced was 148 mg/L. The homodimer percentage was determined according to Example 6. The resulting homodimer percentage was 95.1%, which met the design goal.

The manufacturing yield of the GLP-1-analog-Fc fusion protein of SEQ ID NO: 12 did not reach the target level of 300 mg/L. The manufacturing titer of the GLP-1-analog-Fc fusion protein of SEQ ID NO: 12 was lower than the manufacturing titer of the GLP-1-analog-Fc fusion protein of SEQ ID NO: 11 which included the feline thrombin signal peptide sequence.

The affinity between the GLP-1-analog-Fc fusion protein of SEQ ID NO: 12 and the GLP-1 receptor was measured in a GLP-1R ELISA according to Example 8. The GLP-1-analog-Fc fusion protein showed strong affinity for the GLP-1R (79 ng/mL EC50), which did not meet the design goal, and which is expected to lead to an unacceptably short pharmacokinetic half-life.

As a fourth attempt, the feline thrombin signal peptide was restored, as the feline thrombin signal peptide sequence also acts as a way to potentially stabilize folding of the molecule and may lead to better manufacturing titers. Mutations were made to the feline thrombin signal peptide leader sequence from the prior art (SEQ ID NO: 7) to remove the “RR” site with R18H and R19D mutations, which should prevent potential enzymatic cleavage of the thrombin peptide from the GLP-1-analog-Fc fusion protein in vivo and hopefully retain the desired lower GLP-1R binding for desired long pharmacokinetic half-life in vivo. The resulting mutated feline thrombin signal peptide sequence is given in SEQ ID NO: 13 below.

(SEQ ID NO: 13)
QHVFLAPQQALSLLQRVHD.

The mutated feline thrombin signal peptide sequence of SEQ ID NO: 13 was combined with the DPP-IV resistant GLP-1-analog with mutations including protection from DPP-IV inactivation (A2G), increased solubility (G16E), and reduction of immunogenicity via substituting a glycine residue for arginine (R30G) to remove a potential T-cell epitope. The GLP-1-analog is shown below in SEQ ID NO: 4.

(SEQ ID NO: 4)
HGEGTFTSDVSSYLEEQAAKEFIAWLVKGGG.

The resulting GLP-1-analog is shown below as SEQ ID NO: 14.

(SEQ ID NO: 14)
QHVFLAPQQALSLLQRVHDHGEGTFTSDVSSYLEEQAAKEFIAWLVKG
GG.

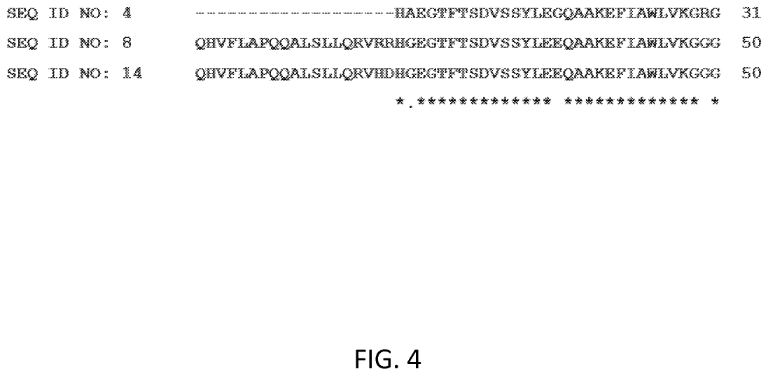

FIG. 4 illustrates a side-by-side sequence comparison of the GLP-1-analogs of SEQ ID NO: 4, SEQ ID NO: 8 and SEQ ID NO: 14. “*” represents complete homology across all sequences at a given sequence position, while “:”, “.” or spaces refer to conservative, moderate, or very different amino acid mutations across the sequences at a given sequence position, respectively.

The feline IgG1b Fc fragment was also mutated to prevent glycosylation. It was speculated that glycan attachment during manufacturing may be negatively impacting the manufacturing yield, and N-linked glycan on the Fc may lead to strong binding to the Fc(gamma) receptors that may lead to increased immune cell activation, increased antigen presentation and unwanted in vivo immunogenicity. The mutation to prevent glycosylation involves mutating the Fc fragment cNg site to prevent glycosylation during synthesis in the host cell. Therefore, cNg site mutations were made to the Fc fragment region of SEQ ID NO: 10 to reduce the binding affinity of the Fc fragment for Fc(gamma) receptors in vivo, as measured by binding in an in vitro human Fc(gamma)R assay described in Example 7. The feline IgG1b Fc fragment with a cNg mutation to serine (S) to prevent glycosylation during manufacturing (SEQ ID NO: 15) is shown below.

(SEQ ID NO: 15)
DCPKCPPPEMLGGPSIFIFPPKPKDTLSISRTPEVTCLVVDLGPDDSDV
QITWFVDNTQVYTAKTSPREEQFSSTYRVVSVLPILHQDWLKGKEFKCK
VNSKSLPSPIERTISKDKGQPHEPQVYVLPPAQEELSRNKVSVTCLIEG
FYPSDIAVEWEITGQPEPENNYRTTPPQLDSDGTYFLYSRLSVDRSRWQ
RGNTYTCSVSHEALHSHHTQKSLTQSPG.

The GLP-1-analog of SEQ ID NO: 14 was linked via the linker GGGGSGGGGSGGGGS (SEQ ID NO: 6) to the feline IgG1b Fc fragment of SEQ ID NO: 15. The resultant GLP-1-analog-Fc fusion protein is given below as SEQ ID NO: 16. The mutation to prevent glycosylation of the Fc fragment is cNg-N138-S.

(SEQ ID NO: 16)
QHVFLAPQQALSLLQRVHDHGEGTFTSDVSSYLEEQAAKEFIAWLVKGG
GGGGGSGGGGSGGGGSDCPKCPPPEMLGGPSIFIFPPKPKDTLSISRTP
EVTCLVVDLGPDDSDVQITWFVDNTQVYTAKTSPREEQFSSTYRVVSVL
PILHQDWLKGKEFKCKVNSKSLPSPIERTISKDKGQPHEPQVYVLPPAQ
EELSRNKVSVTCLIEGFYPSDIAVEWEITGQPEPENNYRTTPPQLDSDG
TYFLYSRLSVDRSRWQRGNTYTCSVSHEALHSHHTQKSLTQSPG.

The GLP-1-analog-Fc fusion protein of SEQ ID NO: 16 was manufactured in CHO cells according to Example 1 and was purified according to Example 3. The Fc fusion protein structure of the GLP-1-analog-Fc fusion protein of SEQ ID NO: 16 was confirmed according to Example 4, and sequence identification is performed according to Example 5. The resultant yield of the GLP-1-analog-Fc fusion protein produced was 427 mg/L. The manufacturing yield of the GLP-1-analog-Fc fusion protein of SEQ ID NO: 16 exceeded the target level of 300 mg/L. The homodimer percentage was determined according to Example 6. The resulting homodimer percentage was 87.8%, which met the design goal.

The affinity between the GLP-1-analog-Fc fusion protein of SEQ ID NO: 16 and the GLP-1 receptor was measured in a GLP-1R ELISA according to Example 8. The GLP-1-analog-Fc fusion protein showed weaker affinity for the GLP-1R than previous sequences (630 ng/mL EC50), which is weak enough to potentially extend the pharmacokinetic half-life of the compound but strong enough to be efficacious and meets the design goal.

Although the GLP-1-analog-Fc fusion protein of SEQ ID NO: 16 met the design goals, it was not known what the primary driver of the reduced GLP-1R affinity necessary to meet the design goals was: the feline thrombin signal peptide mutations of SEQ ID NO: 13 to prevent the enzymatic thrombin cleavage, or the Fc fragment cNg-N73-S mutation of SEQ ID NO: 15 to prevent glycosylation.

As a next step, the feline thrombin signal peptide was restored to its native sequence while retaining the cNg mutation to the glycosylation site of the Fc fragment. The GLP-1-analog sequence utilized a DPP-IV resistant GLP-1-analog which was expected to prolong the structural half-life by preventing in vivo enzymatic clipping of the GLP-1-analog. The DPP-IV resistant GLP-1-analog is shown as SEQ ID NO: 4 below. As shown in FIG. 3, the GLP-1-analog of SEQ ID NO: 4 includes three mutations from the native feline GLP-1(7-37) of SEQ ID NO: 3 in an attempt to improve the pharmacokinetic half-life.

(SEQ ID NO: 4)
HGEGTFTSDVSSYLEEQAAKEFIAWLVKGGG.

The feline thrombin signal peptide (without mutations) as a leader sequence was added and was expected to allow for thrombin enzymatic cleavage in vivo to expose the GLP-1 portion of the molecule, which was used in the prior art sequence as it appeared to confer advantages to the expression of the feline GLP-1-Fc in vivo. The feline thrombin signal peptide leader sequence is shown as SEQ ID NO: 7 below.

(SEQ ID NO: 7)
QHVFLAPQQALSLLQRVRR.

These two sequences combined comprise the GLP-1-analog used in the GLP-1-analog-Fc fusion protein. The GLP-1-analog is shown as SEQ ID NO: 8 below.

(SEQ ID NO: 8)
QHVFLAPQQALSLLQRVRRHGEGTFTSDVSSYLEEQAAKEFIAWLVKGGG.

The feline IgG1b Fc fragment was again mutated to prevent glycosylation. It was speculated that glycan attachment during manufacturing may be negatively impacting the manufacturing yield, and N-linked glycan on the Fc may lead to strong binding to the Fc(gamma) receptors that may lead to increased immune cell activation, increased antigen presentation and unwanted in vivo immunogenicity. The feline IgG1b Fc fragment with a cNg mutation to serine (S) (cNg-N73-S) to prevent glycosylation during manufacturing (SEQ ID NO: 15) is shown below.

(SEQ ID NO: 15)
DCPKCPPPEMLGGPSIFIFPPKPKDTLSISRTPEVTCLVVDLGPDDSDVQ
ITWFVDNTQVYTAKTSPREEQFSSTYRVVSVLPILHQDWLKGKEFKCKVN
SKSLPSPIERTISKDKGQPHEPQVYVLPPAQEELSRNKVSVTCLIEGFYP
SDIAVEWEITGQPEPENNYRTTPPQLDSDGTYFLYSRLSVDRSRWQRGNT
YTCSVSHEALHSHHTQKSLTQSPG.

The GLP-1-analog of SEQ ID NO: 8 was linked via the linker GGGGSGGGGSGGGGS (SEQ ID NO: 6) to the feline IgG1b Fc fragment of SEQ ID NO: 15. The resultant GLP-1-analog-Fc fusion protein is given below as SEQ ID NO: 17. The mutation to prevent glycosylation of the Fc fragment is cNg-N138-S.

(SEQ ID NO: 17)
QHVFLAPQQALSLLQRVRRHGEGTFTSDVSSYLEEQAAKEFIAWLVKGGG
GGGGSGGGGSGGGGSDCPKCPPPEMLGGPSIFIFPPKPKDTLSISRTPEV
TCLVVDLGPDDSDVQITWFVDNTQVYTAKTSPREEQFSSTYRVVSVLPIL
HQDWLKGKEFKCKVNSKSLPSPIERTISKDKGQPHEPQVYVLPPAQEELS
RNKVSVTCLIEGFYPSDIAVEWEITGQPEPENNYRTTPPQLDSDGTYFLY
SRLSVDRSRWQRGNTYTCSVSHEALHSHHTQKSLTQSPG.

The GLP-1-analog-Fc fusion protein of SEQ ID NO: 17 was manufactured in CHO cells according to Example 1 and was purified according to Example 3. The Fc fusion protein structure of the GLP-1-analog-Fc fusion protein of SEQ ID NO: 17 was confirmed according to Example 4, and sequence identification is performed according to Example 5. The resultant yield of the GLP-1-analog-Fc fusion protein produced was 300 mg/L. The manufacturing yield of the GLP-1-analog-Fc fusion protein of SEQ ID NO: 17 just met the target level of 300 mg/L. The homodimer percentage was determined according to Example 6. The resulting homodimer percentage was 87.4%, which met the design goal.

The affinity between the GLP-1-analog-Fc fusion protein of SEQ ID NO: 17 and the GLP-1 receptor was measured in a GLP-1R ELISA according to Example 8. The GLP-1-analog-Fc fusion protein showed slightly stronger affinity for the GLP-1R than the previous sequence with the mutations to the feline thrombin signal peptide leader sequence. The affinity for the GLP-1 receptor as measured in a GLP-1R ELISA (EC50) was 468 ng/mL, which was expected to be weak enough to potentially extend the pharmacokinetic half-life of the compound but strong enough to be efficacious and meet the design goal.

The GLP-1-analog-Fc fusion protein of SEQ ID NO: 17 only just met the manufacturing titer goal of 300 mg/L. Unexpectedly, the GLP-1R receptor binding affinity was only slightly stronger than that of the GLP-1-analog-Fc fusion protein of SEQ ID NO: 16 which had the feline thrombin signal peptide mutated to prevent thrombin enzymatic clipping. A further attempt was tried with a different mutation to the Fc fragment (while maintaining the feline thrombin signal peptide in its native state) in an attempt to further reduce the GLP-1R affinity, which is expected to improve the in vivo pharmacokinetic half-life, and in an attempt to improve the manufacturing yield.

The GLP-1-analog-Fc fusion protein of SEQ ID NO: 17 is administered to N=3 cats according to Example 12 and the pharmacokinetics of the GLP-1-analog-Fc fusion protein of SEQ ID NO: 17 in vivo are measured according to Example 11. The level of the GLP-1-analog-Fc fusion protein measured in serum over time is expected to demonstrate that the GLP-1-analog-Fc fusion protein of SEQ ID NO: 17 will exhibit a pharmacokinetic half-life of greater than 72 hours (3 days), which would meet the design goal for pharmacokinetic half-life.

As a next step, the native feline thrombin signal peptide of SEQ ID NO: 7 was retained, as was the DPP-IV resistant GLP-1-analog which was expected to prolong the structural half-life by preventing in vivo enzymatic clipping of the GLP-1-analog. The DPP-IV resistant GLP-1-analog is shown as SEQ ID NO: 4 below. As shown in FIG. 3, the GLP-1-analog of SEQ ID NO: 4 includes three mutations from the native feline GLP-1(7-37) of SEQ ID NO: 3 in an attempt to improve the structural half-life.

(SEQ ID NO: 4)
HGEGTFTSDVSSYLEEQAAKEFIAWLVKGGG.

The feline thrombin signal peptide (without mutations) as a leader sequence was added and was expected to allow for thrombin enzymatic cleavage in vivo to expose the GLP-1 portion of the molecule, which was used in the prior art sequence as it appeared to confer advantages to the expression of the feline GLP-1-Fc in vivo. The feline thrombin signal peptide leader sequence is shown as SEQ ID NO: 7 below.

(SEQ ID NO: 7)
QHVFLAPQQALSLLQRVRR.

These two sequences combined comprise the GLP-1-analog used in the GLP-1-analog-Fc fusion protein. The GLP-1-analog is shown as SEQ ID NO: 8 below.

(SEQ ID NO: 8)
QHVFLAPQQALSLLQRVRRHGEGTFTSDVSSYLEEQAAKEFIAWLVKGGG.

The feline IgG1b Fc fragment was again mutated to prevent glycosylation. It was speculated that glycan attachment during manufacturing may be negatively impacting the manufacturing yield. Additionally, the mutation of the glycosylation site will minimize Fc(gamma)R binding which is expected to reduce unwanted immunogenicity. A different mutation was attempted in an effort to improve the manufacturing yield and retain or weaken the GLP-1R binding affinity as compared to that of SEQ ID NO: 17. The feline IgG1b Fc fragment with a cNg mutation to glutamine (Q) to prevent glycosylation during manufacturing (SEQ ID NO: 18) is shown below.

(SEQ ID NO: 18)
DCPKCPPPEMLGGPSIFIFPPKPKDTLSISRTPEVTCLVVDLGPDDSDVQ
ITWFVDNTQVYTAKTSPREEQFQSTYRVVSVLPILHQDWLKGKEFKCKVN
SKSLPSPIERTISKDKGQPHEPQVYVLPPAQEELSRNKVSVTCLIEGFYP
SDIAVEWEITGQPEPENNYRTTPPQLDSDGTYFLYSRLSVDRSRWQRGNT
YTCSVSHEALHSHHTQKSLTQSPG.

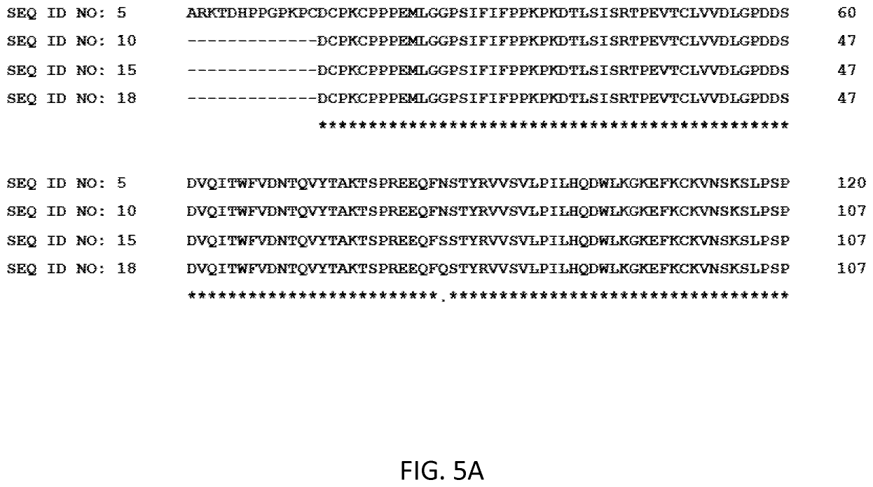

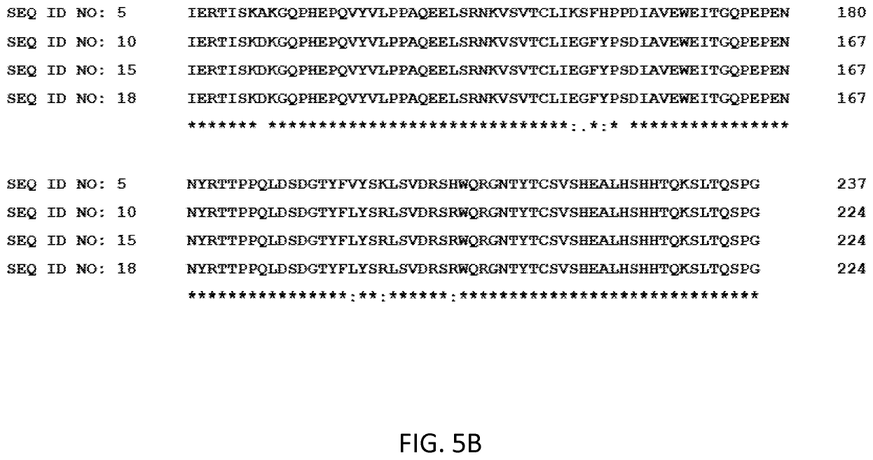

FIG. 5A and FIG. 5B illustrate a side-by-side sequence comparison of the Fc fragments of SEQ ID NO: 5, SEQ ID NO: 10, SEQ ID NO: 15, and SEQ ID NO: 18. “*” represents complete homology across all sequences at a given sequence position, while: “:”, “.” or spaces refer to conservative, moderate, or very different amino acid mutations across the sequences at a given sequence position, respectively.

The GLP-1-analog of SEQ ID NO: 8 was linked via the linker GGGGSGGGGSGGGGS (SEQ ID NO: 6) to the feline IgG1b Fc fragment of SEQ ID NO: 18. The resultant GLP-1-analog-Fc fusion protein is given below as SEQ ID NO: 19. The mutation to prevent glycosylation of the Fc fragment is cNg-N138-Q.

(SEQ ID NO: 19)
QHVFLAPQQALSLLQRVRRHGEGTFTSDVSSYLEEQAAKEFIAWLVKGGG
GGGGSGGGGSGGGGSDCPKCPPPEMLGGPSIFIFPPKPKDTLSISRTPEV
TCLVVDLGPDDSDVQITWFVDNTQVYTAKTSPREEQFQSTYRVVSVLPIL
HQDWLKGKEFKCKVNSKSLPSPIERTISKDKGQPHEPQVYVLPPAQEELS
RNKVSVTCLIEGFYPSDIAVEWEITGQPEPENNYRTTPPQLDSDGTYFLY
SRLSVDRSRWQRGNTYTCSVSHEALHSHHTQKSLTQSPG.

The GLP-1-analog-Fc fusion protein of SEQ ID NO: 19 was manufactured in CHO cells according to Example 1 and was purified according to Example 3. The Fc fusion protein structure of the GLP-1-analog-Fc fusion protein of SEQ ID NO: 19 was confirmed according to Example 4, and sequence identification was performed according to Example 14. The resultant yield of the GLP-1-analog-Fc fusion protein produced was 507 mg/L. The manufacturing yield of the GLP-1-analog-Fc fusion protein of SEQ ID NO: 19 exceeded the target level of 300 mg/L. The homodimer percentage was determined according to Example 6. The resulting homodimer percentage was 88.6%, which also met the design goal.

The affinity between the GLP-1-analog-Fc fusion protein of SEQ ID NO: 19 and the GLP-1 receptor was measured in a GLP-1R ELISA according to Example 8. As with the previous sequence (SEQ ID NO: 17) which similarly included a mutation to the natural glycosylation site of the feline Fc fragment, unexpectedly, a minor change to the feline Fc fragment (cNg-N138-Q in SEQ ID NO: 19) led to a significant impact to the GLP-1R binding affinity. The GLP-1-analog-Fc fusion protein of SEQ ID NO: 19 showed similar affinity for the GLP-1R as the previous GLP-1-analog-Fc fusion protein (SEQ ID NO: 17). The affinity for the GLP-1 receptor as measured in a GLP-1R ELISA (EC50) was 458 ng/mL, which was expected to be weak enough to potentially extend the pharmacokinetic half-life of the compound but strong enough to be efficacious and meet the design goal.

The change to the feline Fc fragment (cNg-N138-Q in SEQ ID NO: 19) additionally resulted in a significant improvement in manufacturability, and the mutation of the glycosylation site will minimize Fc(gamma)R binding which is expected to reduce unwanted immunogenicity. The GLP-1-analog-Fc fusion protein of SEQ ID NO: 19 met the manufacturing titer target (507 mg/L) and also had an acceptable GLP-1R binding of 458 ng/mL.

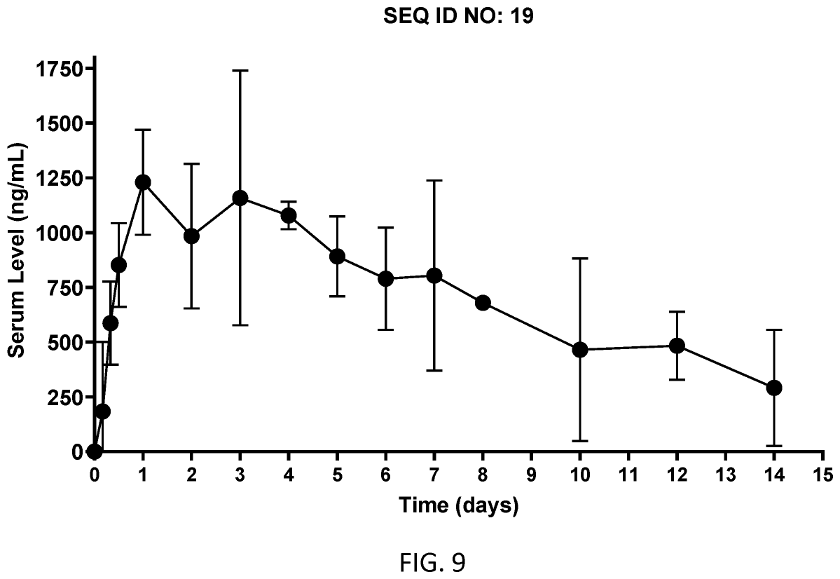

The GLP-1-analog-Fc fusion protein of SEQ ID NO: 19 was administered to N=3 cats according to Example 10. and the pharmacokinetics of the GLP-1-analog-Fc fusion protein of SEQ ID NO: 19 in vivo was measured according to Example 9. The level of the GLP-1-analog-Fc fusion protein measured in serum over time is shown in FIG. 9, demonstrating that the GLP-1-analog-Fc fusion protein of SEQ ID NO: 19 exhibited a pharmacokinetic half-life of 161 hours (6.7 days), which met the design goal.

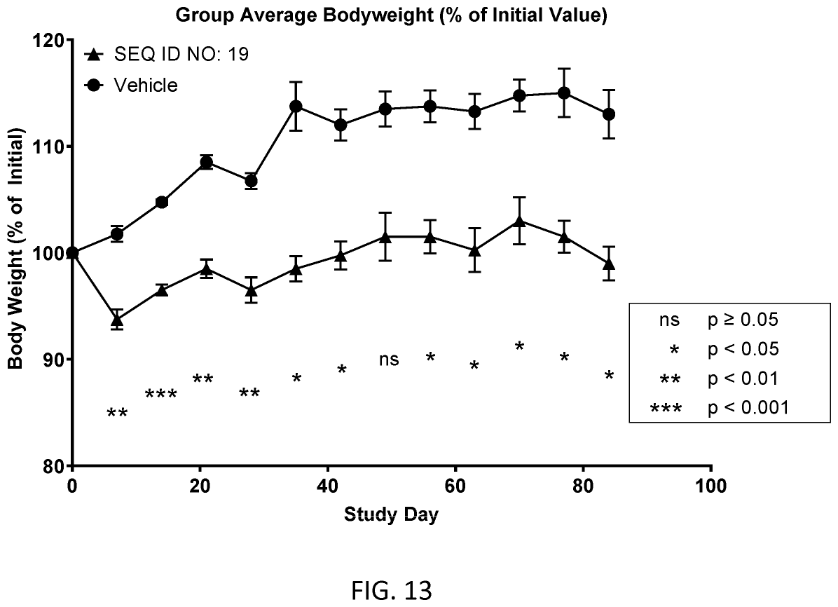

The GLP-1-analog-Fc fusion protein of SEQ ID NO: 19 was administered to N=4 cats according to Example 15, and cats' body weights were measured according to Example 15. FIG. 13 illustrates the observed body weight, as a percentage of initial measured body weight, of cats (N=4) treated with weekly injections of the GLP-1-analog-Fc fusion protein of SEQ ID NO: 19 and a control group of cats treated only with injections of vehicle. The number of asterisks at each measurement day along the horizontal axis indicates an upper bound for the statistical p-value comparing the SEQ ID NO: 19 group to the vehicle group, where “ns” indicates that p≥0.05, “*” indicates that p<0.05, “**” indicates that p<0.01, and “***” indicates that p<0.001. FIG. 13 demonstrates that the administration of the GLP-1-analog-Fc fusion protein of SEQ ID NO: 19 had a significant measurable impact on reducing food intake and observed weight gain, therefore leading to leaner cats relative to the control group.

Although the fusion protein of SEQ ID NO: 16 met the design goals, it was not known what the primary driver of the reduced GLP-1R affinity necessary to meet the design goals was: the feline thrombin signal peptide mutations to prevent the enzymatic thrombin cleavage, or the Fc fragment mutation to prevent glycosylation. A further attempt was tried with a different mutation to the Fc fragment (while maintaining the feline thrombin signal peptide mutations) in an attempt to further reduce the GLP-1R affinity, which is expected to improve the in vivo half-life.

As a next step, the mutated feline thrombin signal peptide of SEQ ID NO: 13 was considered again, as was the DPP-IV resistant GLP-1-analog which was expected to prolong the structural half-life by preventing in vivo enzymatic clipping of the GLP-1-analog. The DPP-IV resistant GLP-1-analog is shown as SEQ ID NO: 4 below. As shown in FIG. 3, the GLP-1-analog of SEQ ID NO: 4 includes three mutations from the native feline GLP-1(7-37) of SEQ ID NO: 3 in an attempt to improve the structural half-life.

(SEQ ID NO: 4)
HGEGTFTSDVSSYLEEQAAKEFIAWLVKGGG.

The mutated feline thrombin signal peptide as a leader sequence was added, where the mutations were expected to prevent thrombin enzymatic cleavage in vivo. The feline thrombin signal peptide leader sequence is shown as SEQ ID NO: 13 below.

(SEQ ID NO: 13)
QHVFLAPQQALSLLQRVHD.

These two sequences combined comprise the GLP-1-analog used in the GLP-1-analog-Fc fusion protein. The GLP-1-analog is shown as SEQ ID NO: 14 below.

(SEQ ID NO: 14)
QHVFLAPQQALSLLQRVHDHGEGTFTSDVSSYLEEQAAKEFIAWLVKGGG.

The feline IgG1b Fc fragment was again mutated to prevent glycosylation. It was speculated that glycan attachment during manufacturing may be negatively impacting the manufacturing yield. A different mutation was attempted in an effort to improve the manufacturing yield and retain or weaken the GLP-1R binding affinity as compared to that of SEQ ID NO: 16. The feline IgG1b Fc fragment with a cNg mutation to glutamine (Q) to prevent glycosylation during manufacturing (SEQ ID NO: 18) is shown below.

(SEQ ID NO: 18)
DCPKCPPPEMLGGPSIFIFPPKPKDTLSISRTPEVTCLVVDLGPDDSDVQ
ITWFVDNTQVYTAKTSPREEQFQSTYRVVSVLPILHQDWLKGKEFKCKVN
SKSLPSPIERTISKDKGQPHEPQVYVLPPAQEELSRNKVSVTCLIEGFYP
SDIAVEWEITGQPEPENNYRTTPPQLDSDGTYFLYSRLSVDRSRWQRGNT
YTCSVSHEALHSHHTQKSLTQSPG.

The GLP-1-analog of SEQ ID NO: 14 was linked via the linker GGGGSGGGGSGGGGS (SEQ ID NO: 6) to the feline IgG1b Fc fragment of SEQ ID NO: 18. The resultant GLP-1-analog-Fc fusion protein is given below as SEQ ID NO: 20. The mutation to prevent glycosylation of the Fc fragment is cNg-N138-Q.

(SEQ ID NO: 20)
QHVFLAPQQALSLLQRVHDHGEGTFTSDVSSYLEEQAAKEFIAWLVKGGG
GGGGSGGGGSGGGGSDCPKCPPPEMLGGPSIFIFPPKPKDTLSISRTPEV
TCLVVDLGPDDSDVQITWFVDNTQVYTAKTSPREEQFQSTYRVVSVLPIL
HQDWLKGKEFKCKVNSKSLPSPIERTISKDKGQPHEPQVYVLPPAQEELS
RNKVSVTCLIEGFYPSDIAVEWEITGQPEPENNYRTTPPQLDSDGTYFLY
SRLSVDRSRWQRGNTYTCSVSHEALHSHHTQKSLTQSPG.

The GLP-1-analog-Fc fusion protein of SEQ ID NO: 20 was manufactured in CHO cells according to Example 1 and was purified according to Example 3. The Fc fusion protein structure of the GLP-1-analog-Fc fusion protein of SEQ ID NO: 20 was confirmed according to Example 4, and sequence identification is performed according to Example 5. The resultant yield of the GLP-1-analog-Fc fusion protein produced was 611 mg/L. The manufacturing yield of the GLP-1-analog-Fc fusion protein of SEQ ID NO: 20 exceeded the target level of 300 mg/L. The homodimer percentage was determined according to Example 6. The resulting homodimer percentage was 89.2%, which also met the design goal.

The affinity between the GLP-1-analog-Fc fusion protein of SEQ ID NO: 20 and the GLP-1 receptor was measured in a GLP-1R ELISA according to Example 8. Unexpectedly, a minor change to the feline Fc fragment (cNg-N138-Q in SEQ ID NO: 20) led to a significant impact to the GLP-1R binding affinity, even as compared to the previously-evaluated sequence of SEQ ID NO: 16 which similarly included a mutation (cNg-N138-S) to the natural glycosylation site of the feline Fc fragment. The GLP-1-analog-Fc fusion protein of SEQ ID NO: 20 showed significantly weaker affinity for the GLP-1R as compared to the previously-evaluated GLP-1-analog-Fc fusion protein of SEQ ID NO: 16. The affinity for the GLP-1 receptor as measured in a GLP-1R ELISA (EC50) for SEQ ID NO: 20 was 1651 ng/mL, which was expected to be weak enough to potentially extend the pharmacokinetic half-life of the compound but strong enough to be efficacious and meet the design goal.

The change to the feline Fc fragment (cNg-N138-Q in SEQ ID NO: 20) additionally resulted in a significant improvement in manufacturability. The GLP-1-analog-Fc fusion protein of SEQ ID NO: 20 met the manufacturing titer target with a titer of 611 mg/L and also had an acceptable GLP-1R binding affinity EC50 of 1651 ng/mL.

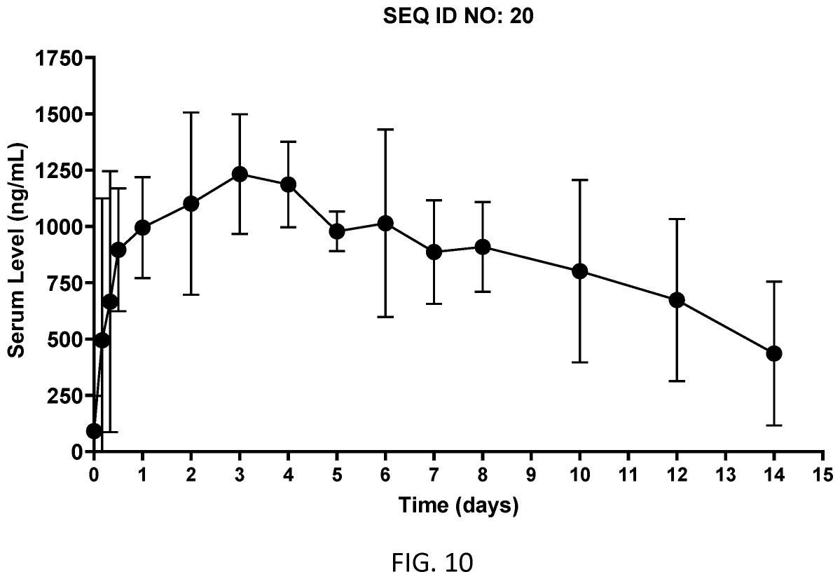

The GLP-1-analog-Fc fusion protein of SEQ ID NO: 20 was administered to N=3 cats according to Example 10 and the pharmacokinetics of the GLP-1-analog-Fc fusion protein of SEQ ID NO: 20 in vivo was measured according to Example 9. The level of the GLP-1-analog-Fc fusion protein measured in serum over time is shown in FIG. 10, demonstrating that the GLP-1-analog-Fc fusion protein of SEQ ID NO: 20 exhibited a pharmacokinetic half-life of 204 hours (8.5 days), which met the design goal.

The calculated MW, manufacturing titers and 0% homodimer of the GLP-1-analog-Fc fusion proteins of SEQ ID NO: 9, SEQ ID NO: 11, SEQ ID NO: 12, SEQ ID NO: 16, SEQ ID NO: 17, SEQ ID NO: 19 and SEQ ID NO: 20 are shown below in Table 1.

TABLE 1
Manufacturing titers of GLP-1-analog-Fc fusion proteins

| | Calc. MW, without | | |
|---|---|---|---|
| Sequence | glycan (Da) | Titer (mg/L) | % Homodimer |
| SEQ ID NO: 9 | 66154.28 | 569 | 81.6 |
| SEQ ID NO: 11 | 63569.08 | 252 | 85.5 |
| SEQ ID NO: 12 | 59083.76 | 148 | 95.1 |
| SEQ ID NO: 16 | 63394.74 | 427 | 87.8 |
| SEQ ID NO: 17 | 63515.02 | 300 | 87.4 |
| SEQ ID NO: 19 | 63597.14 | 507 | 88.6 |
| SEQ ID NO: 20 | 63597.14 | 611 | 89.2 |

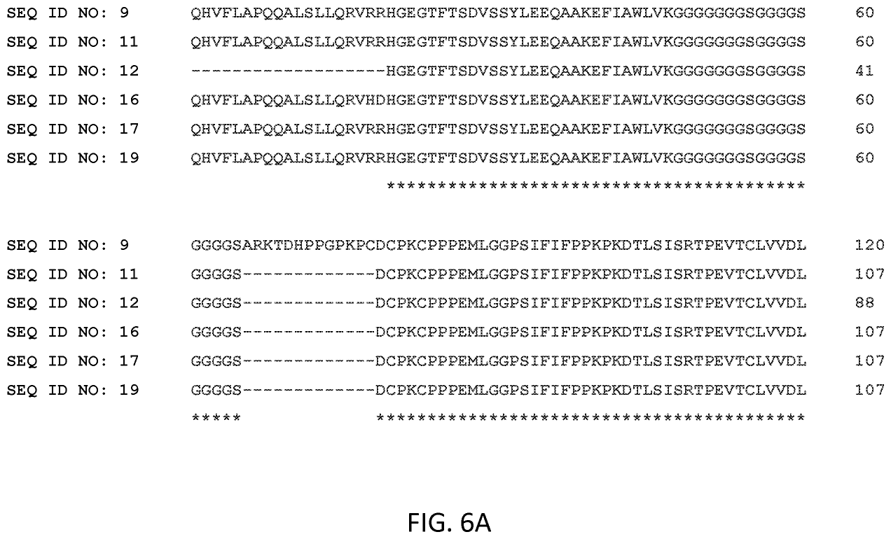

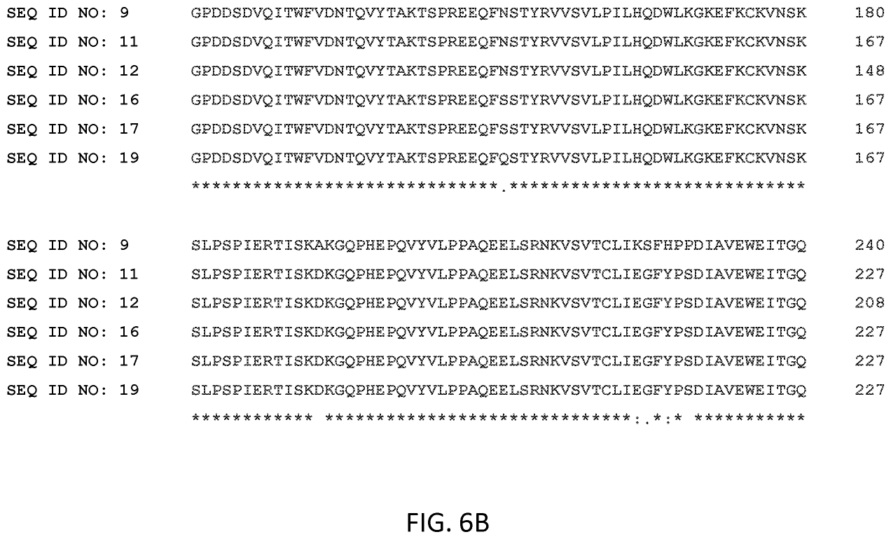

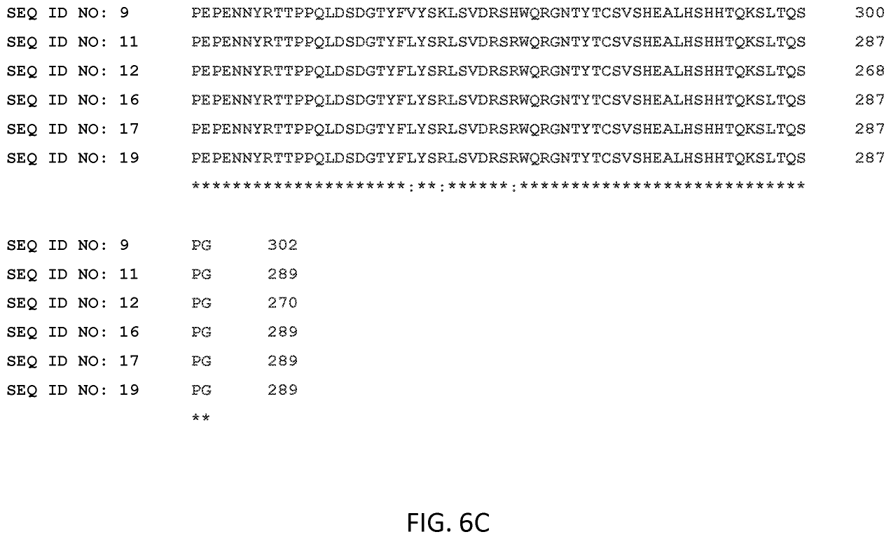

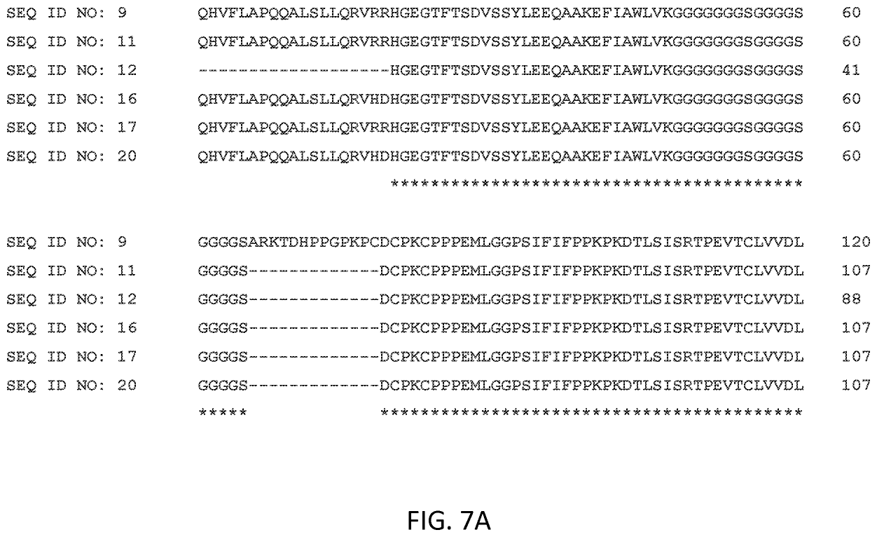

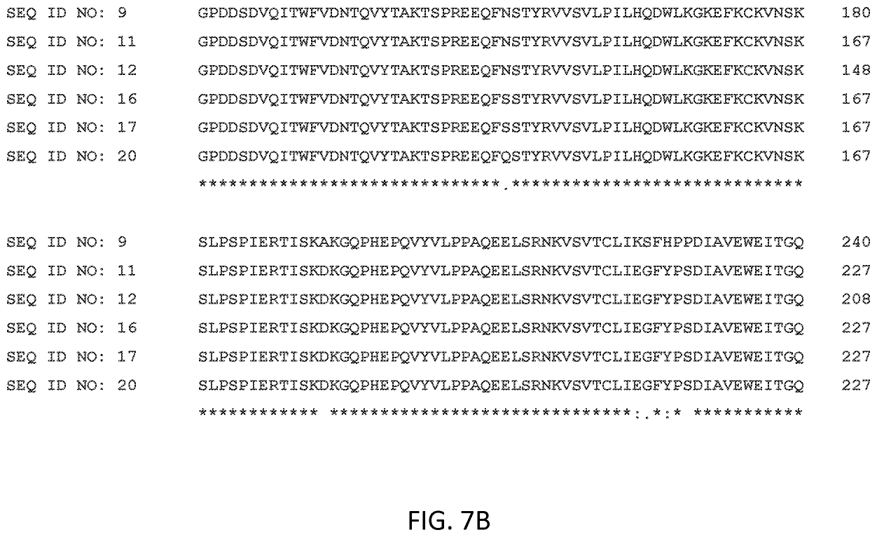

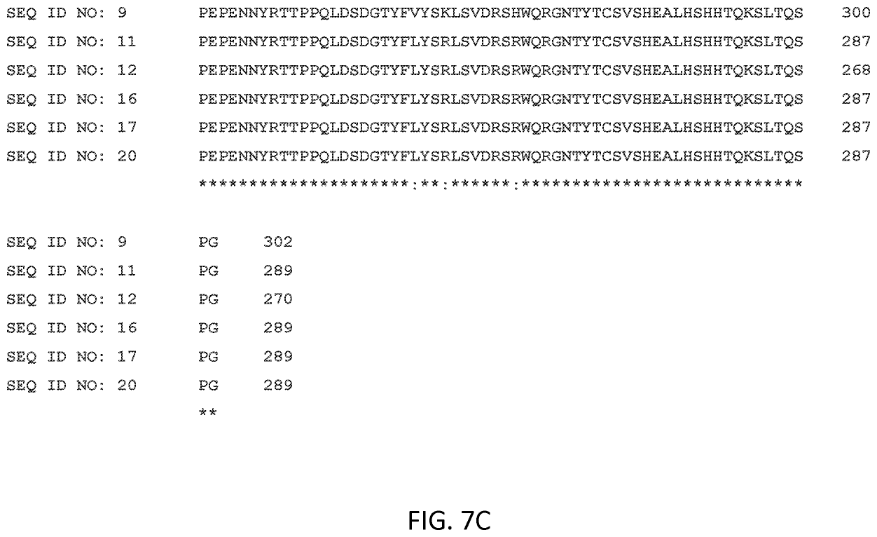

A sequence comparison of the GLP-1-analog-Fc fusion proteins of SEQ ID NO: 9, SEQ ID NO: 11, SEQ ID NO: 12, SEQ ID NO: 16, SEQ ID NO: 17 and SEQ ID NO: 19 is shown in FIG. 6A, FIG. 6B3, and FIG. 6C. A sequence comparison of the GLP-1-analog-Fc fusion proteins of SEQ ID NO: 9, SEQ ID NO: 11, SEQ ID NO: 12, SEQ ID NO: 16, SEQ ID NO: 17 and SEQ ID NO: 20 is shown in FIG. 7A. FIG. 7B3, and FIG. 7C.

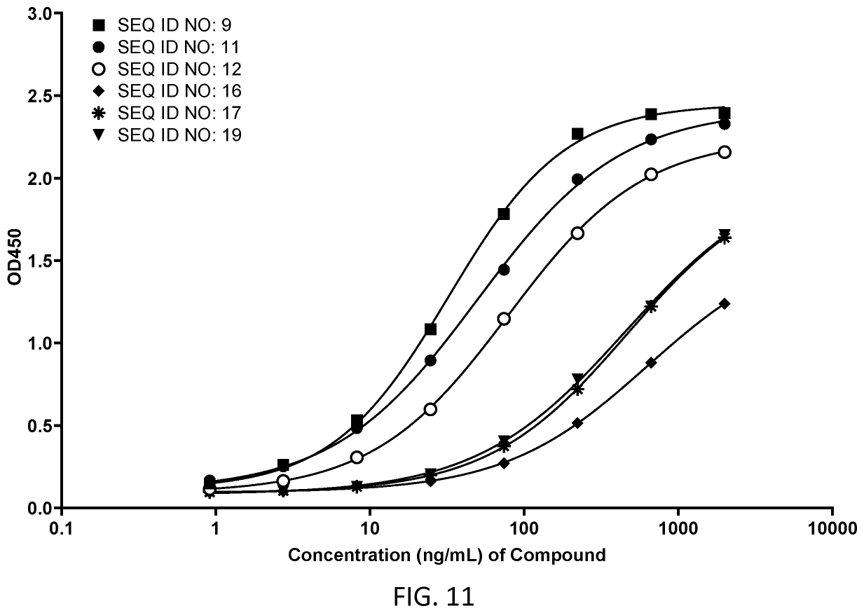

FIG. 11 illustrates the GLP-1-analog-Fc fusion protein in vitro GLP-1R binding affinity at various concentrations of the GLP-1-analog-Fc fusion proteins of SEQ ID NO: 9, SEQ ID NO: 11, SEQ ID NO: 12, SEQ ID NO: 16, SEQ ID NO: 17, and SEQ ID NO: 19. As illustrated in FIG. 7, the GLP-1R binding affinity of the GLP-1-analog-Fc fusion protein of SEQ ID NO: 19 at a concentration of 2000 ng/mL is greater than 0.25 (OD450 at 2000 ng/mL=1.654) and the affinity for the GLP-1 receptor as measured in a GLP-1R ELISA (EC50) was 458 ng/mL, which was expected to be weak enough to potentially extend the pharmacokinetic half-life of the compound but strong enough to be efficacious and meet the design goal.

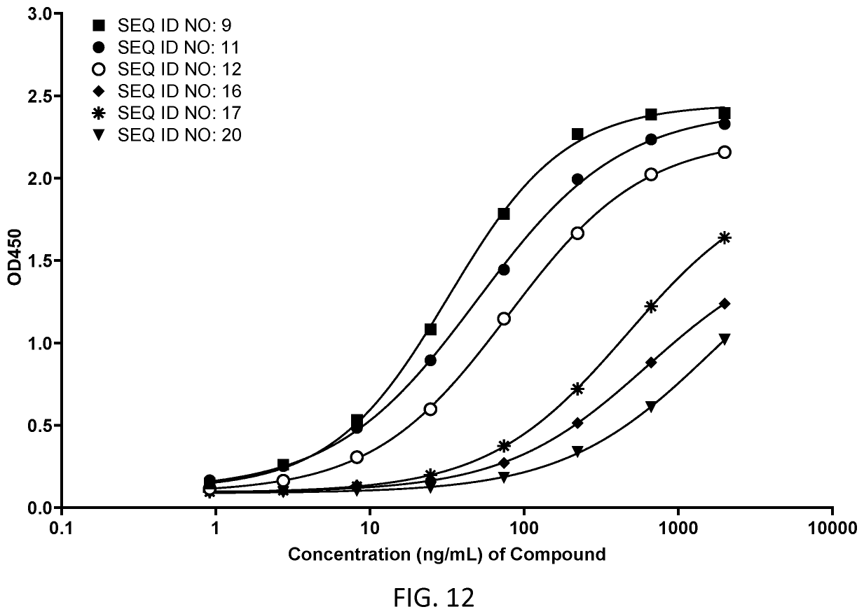

FIG. 12 illustrates the GLP-1-analog-Fc fusion protein in vitro GLP-1R binding affinity at various concentrations of the GLP-1-analog-Fc fusion proteins of SEQ ID NO: 9, SEQ ID NO: 11, SEQ ID NO: 12, SEQ ID NO: 16, SEQ ID NO: 17, and SEQ ID NO: 20. As illustrated in FIG. 7, the GLP-1R binding affinity of the GLP-1-analog-Fc fusion protein of SEQ ID NO: 20 at a concentration of 2000 ng/mL is greater than 0.25 (OD450 at 2000 ng/mL=1.020) and the affinity for the GLP-1 receptor as measured in a GLP-1R ELISA (EC50) was 1651 ng/mL, which was expected to be weak enough to potentially extend the pharmacokinetic half-life of the compound but strong enough to be efficacious and meet the design goal.

### Fusion Protein

Provided herein are fusion proteins, e.g., GLP-1-analog-Fc Fusion proteins. In embodiments, the fusion protein comprises a GLP-1-analog described herein. In embodiments, the fusion protein comprises an Fc fragment, e.g., an Fc fragment described herein. In embodiments, the fusion protein comprises a linker between the GLP-1-analog and the Fc fragment.

In embodiments, the GLP-1-analog is located on the N-terminal side of the Fc fragment. In embodiments, the fusion protein comprises domains in the following orientation from N- to C-termini: (N-terminus)-GLP-1-analog polypeptide-linker-Fc fragment (C-terminus) as illustrated in FIG. 1. In embodiments, the fusion protein, also referred to as the GLP-1-analog-Fc fusion protein, is comprised of two identical GLP-1-analog-Fc fusion proteins covalently bound together via one or more disulfide bonds (shown as dotted lines in FIG. 1; total number of disulfide bonds in actuality may be greater or lower than the number shown in FIG. 1). Each GLP-1-analog-Fc fusion protein comprises a GLP-1-analog polypeptide molecule and a linker, where the GLP-1-analog-Fc fusion protein sequence terminates in the Fc-CH3-C-terminal region.

The full-length sequences of the GLP-1-analog-Fc fusion proteins of the present technology are provided below.

```
(SEQ ID NO: 9)
QHVFLAPQQALSLLQRVRRHGEGTFTSDVSSYLEEQAAKEFIAWLVKGGG
GGGGSGGGGSGGGGSARKTDHPPGPKPCDCPKCPPPEMLGGPSIFIFPPK
PKDTLSISRTPEVTCLVVDLGPDDSDVQITWFVDNTQVYTAKTSPREEQF
NSTYRVVSVLPILHQDWLKGKEFKCKVNSKSLPSPIERTISKAKGQPHEP
QVYVLPPAQEELSRNKVSVTCLIKSFHPPDIAVEWEITGQPEPENNYRTT
PPQLDSDGTYFVYSKLSVDRSHWQRGNTYTCSVSHEALHSHHTQKSLTQS
PG.
  (SEQ ID NO: 11)
QHVFLAPQQALSLLQRVRRHGEGTFTSDVSSYLEEQAAKEFIAWLVKGGG
GGGGSGGGGSGGGGSDCPKCPPPEMLGGPSIFIFPPKPKDTLSISRTPEV
TCLVVDLGPDDSDVQITWFVDNTQVYTAKTSPREEQFNSTYRVVSVLPIL
HQDWLKGKEFKCKVNSKSLPSPIERTISKDKGQPHEPQVYVLPPAQEELS
RNKVSVTCLIEGFYPSDIAVEWEITGQPEPENNYRTTPPQLDSDGTYFLY
SRLSVDRSRWQRGNTYTCSVSHEALHSHHTQKSLTQSPG.
  (SEQ ID NO: 12)
HGEGTFTSDVSSYLEEQAAKEFIAWLVKGGGGGGGSGGGGSGGGGSDCPK
CPPPEMLGGPSIFIFPPKPKDTLSISRTPEVTCLVVDLGPDDSDVQITWF
VDNTQVYTAKTSPREEQFNSTYRVVSVLPILHQDWLKGKEFKCKVNSKSL
PSPIERTISKDKGQPHEPQVYVLPPAQEELSRNKVSVTCLIEGFYPSDIA
VEWEITGQPEPENNYRTTPPQLDSDGTYFLYSRLSVDRSRWQRGNTYTCS
VSHEALHSHHTQKSLTQSPG.
  (SEQ ID NO: 16)
QHVFLAPQQALSLLQRVHDHGEGTFTSDVSSYLEEQAAKEFIAWLVKGGG
GGGGSGGGGSGGGGSDCPKCPPPEMLGGPSIFIFPPKPKDTLSISRTPEV
TCLVVDLGPDDSDVQITWFVDNTQVYTAKTSPREEQFSSTYRVVSVLPIL
HQDWLKGKEFKCKVNSKSLPSPIERTISKDKGQPHEPQVYVLPPAQEELS
RNKVSVTCLIEGFYPSDIAVEWEITGQPEPENNYRTTPPQLDSDGTYFLY
SRLSVDRSRWQRGNTYTCSVSHEALHSHHTQKSLTQSPG.
  (SEQ ID NO: 17)
QHVFLAPQQALSLLQRVRRHGEGTFTSDVSSYLEEQAAKEFIAWLVKGGG
GGGGSGGGGSGGGGSDCPKCPPPEMLGGPSIFIFPPKPKDTLSISRTPEV
TCLVVDLGPDDSDVQITWFVDNTQVYTAKTSPREEQFSSTYRVVSVLPIL
HQDWLKGKEFKCKVNSKSLPSPIERTISKDKGQPHEPQVYVLPPAQEELS
RNKVSVTCLIEGFYPSDIAVEWEITGQPEPENNYRTTPPQLDSDGTYFLY
SRLSVDRSRWQRGNTYTCSVSHEALHSHHTQKSLTQSPG.
  (SEQ ID NO: 19)
QHVFLAPQQALSLLQRVRRHGEGTFTSDVSSYLEEQAAKEFIAWLVKGGG
GGGGSGGGGSGGGGSDCPKCPPPEMLGGPSIFIFPPKPKDTLSISRTPEV
TCLVVDLGPDDSDVQITWFVDNTQVYTAKTSPREEQFQSTYRVVSVLPIL
HQDWLKGKEFKCKVNSKSLPSPIERTISKDKGQPHEPQVYVLPPAQEELS
RNKVSVTCLIEGFYPSDIAVEWEITGQPEPENNYRTTPPQLDSDGTYFLY
SRLSVDRSRWQRGNTYTCSVSHEALHSHHTQKSLTQSPG.
  (SEQ ID NO: 20)
QHVFLAPQQALSLLQRVHDHGEGTFTSDVSSYLEEQAAKEFIAWLVKGGG
GGGGSGGGGSGGGGSDCPKCPPPEMLGGPSIFIFPPKPKDTLSISRTPEV
TCLVVDLGPDDSDVQITWFVDNTQVYTAKTSPREEQFQSTYRVVSVLPIL
HQDWLKGKEFKCKVNSKSLPSPIERTISKDKGQPHEPQVYVLPPAQEELS
RNKVSVTCLIEGFYPSDIAVEWEITGQPEPENNYRTTPPQLDSDGTYFLY
SRLSVDRSRWQRGNTYTCSVSHEALHSHHTQKSLTQSPG.
```

In some embodiments, the fusion protein is in a preparation. In embodiments, the preparation has a percent dimer, e.g., homodimer, of the fusion protein that is greater than about 65%, (e.g., about 70%, about 75%, about 80% or about 85%). In embodiments, the percent homodimer is about 80% or higher and can be made 85% or higher using one or more processing steps (e.g., ion exchange chromatography, gel filtration, hydrophobic interaction chromatography, etc.). In some embodiments, the % dimer, e.g., homodimer, in the preparation is determined by size-exclusion chromatography which is an analytical separation method that can discriminate between dimers, e.g., homodimers, and higher-order non-covalent Fc fusion protein aggregates (e.g., multimers). In some embodiments, GLP-1-analog-Fc fusion proteins with substantially greater homodimer content than other GLP-1-analog-Fc fusion proteins demonstrate more bioactivity in a subject (e.g., a cat).

### Fusion Protein Production

In embodiments, a fusion protein can be expressed by a vector as described in the Examples section.

In embodiments, a fusion protein can be expressed recombinantly, e.g., in a eukaryotic cell, e.g., mammalian cell or non-mammalian cell. Exemplary mammalian cells used for expression include HEK cells, e.g., HEK293 cells, or CHO cells. In embodiments, cells are transfected with a nucleic acid molecule, e.g., vector, encoding the fusion protein (e.g., where the entire fusion protein is encoded by a single nucleic acid molecule). In other embodiments, cells are transfected with more than one nucleic acid molecule, where each nucleic acid molecule encodes a different domain of the fusion protein. For example, one nucleic acid molecule can encode the GLP-1-analog polypeptide, and a different nucleic acid molecule can encode the Fc fragment. Cells can be cultured using standard methods in the art.

In some embodiments, the fusion protein is purified or isolated from the cells (e.g., by lysis of the cells). In other embodiments, the fusion protein is secreted by the cells and, e.g., the fusion protein is purified or isolated from the cell culture media in which the cells were grown. Purification of the fusion protein can include using column chromatography, e.g., affinity chromatography, or using other separation methods that involve size, charge, and/or affinity for certain molecules. In embodiments, purification of the fusion protein involves selecting or enriching for proteins with an Fc fragment, e.g., by using Protein A beads or a Protein A column that cause proteins containing an Fc fragment to become bound with high affinity at neutral solution pH to the Protein A covalently conjugated to the Protein A beads. The bound Fc fusion protein may then be eluted from the Protein A beads by a change in a solution variable (e.g., a decrease in the solution pH). Other separation methods such as ion exchange chromatography and/or gel filtration chromatography can also be employed alternatively or in addition. In embodiments, purification of the fusion protein further comprises filtering or centrifuging the protein preparation. In embodiments, further purification of the fusion protein comprises diafiltration, ultrafiltration, and filtration through porous membranes of various sizes, as well as final formulation with excipients.

The purified fusion protein can be characterized, e.g., for purity, yield, structure, and/or activity, using a variety of methods, e.g., absorbance at 280 nm (e.g., to determine yield), size exclusion or capillary electrophoresis (e.g., to determine the molecular weight, percent aggregation, and/or purity), mass spectrometry (MS) and/or liquid chromatography (LC-MS) (e.g., to determine purity and/or glycosylation), and/or ELISA (e.g., to determine extent of binding, e.g., affinity, to a GLP-1 receptor). Exemplary methods of characterization are also described in the Examples section.

### Functional Features of Fusion Proteins

Described herein are methods for appetite suppression, for treatment of obesity, for prevention of obesity, or for treatment of other conditions associated with excess weight in cats, the methods comprising the administration of a fusion protein (e.g., a fusion protein described herein) to a subject. In embodiments, a fusion protein described herein is capable of increasing the feelings of satiety resulting in reduction of food intake and consequent weight loss or weight gain prevention. In embodiments, a fusion protein described herein is capable of slowing gastric emptying, meaning food stays in the stomach longer, contributing to prolonged feelings of fullness, reducing hunger. In embodiments, a fusion protein described herein is capable of inhibiting glucagon. In embodiments, a fusion protein described herein is capable of acting both peripherally (in the gut) and centrally (in the brain) to decrease calorie intake and enhance satiety, contributing to weight management. In embodiments, the fusion protein is long-acting (e.g., has a long pharmacokinetic half-life, e.g., in serum). In embodiments, the serum half-life of the fusion protein (e.g., in the blood of a subject upon administration) is longer than about 24 hours. In embodiments, the serum pharmacokinetic half-life of the fusion protein is 3 days, or longer. In embodiments, the serum pharmacokinetic half-life of the fusion protein is longer than that of a native GLP-1(7-37) reference standard or control formulation.

In embodiments, the duration of activity of the fusion protein (e.g., the time during which the fusion protein is detectable in the blood serum of a subject relative to a pre-dose level) is longer than about 24 hours. In embodiments, the duration of activity of the fusion protein (e.g., the time during which the fusion protein is detectable in the blood serum of a subject relative to a pre-dose level) is longer than about 2 days, 2.5 days, 3 days, 4 days, 5, 6 days, 7 days or longer. In embodiments, the duration of activity of the fusion protein (e.g., the time during which the fusion protein is detectable in the blood serum of a subject relative to a pre-dose level) is longer than that of a GLP-1(7-37) reference standard or control formulation.

### Methods of Treatment and Characteristics of Subject Selection

Described herein are methods for interacting with the GLP-1 receptor to suppress appetite, for treatment of obesity, for prevention of obesity, or for treatment of other conditions associated with excess weight in cats, wherein the method comprises administering to a subject a fusion protein, e.g., a fusion protein described herein.

In embodiments, a reference standard used in any method described herein comprises a reference treatment or reference therapy.

### Pharmaceutical Compositions and Routes of Administration

Provided herein are pharmaceutical compositions containing a fusion protein described herein that can be used to lower body weight in cats or to prevent weight gain in cats. The amount and concentration of the fusion protein in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions.

Formulations of the present disclosure include those suitable for parenteral administration. The phrases “parenteral administration” and “administered parenterally” as used herein means modes of administration other than enteral and topical administration, usually by intravenous or subcutaneous injection.

Examples of suitable aqueous and non-aqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, using coating materials, such as lecithin, by the maintenance of the required particle size in the case of dispersions, and using surfactants, e.g., Tween-like surfactants. In some embodiments, the pharmaceutical composition (e.g., as described herein) comprises a Tween-like surfactant, e.g., Tween-20 or Tween-80. In some embodiments, the pharmaceutical composition (e.g., as described herein) comprises a Tween-like surfactant, Tween-like surfactant, e.g., polysorbate-20, Tween-20, or Tween-80, at a concentration between about 0.001% and about 2%, or between about 0.005% and about 0.1%, or between about 0.01% and about 0.5%.

In some embodiments, the fusion protein is administered as a bolus, infusion, or an intravenous push. In some embodiments, the fusion protein is administered through syringe injection, pump, pen, needle, or indwelling catheter. Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

### Dosages

Actual dosage levels of the fusion protein can be varied to obtain an amount of the active ingredient that is effective to achieve the desired therapeutic or prophylactic response for a particular cat. The selected dosage level will depend upon a variety of factors including the activity of the particular fusion protein employed, or the ester, salt or amide thereof, the route of administration, the time of administration, the rate of excretion of the particular compound being employed, the duration of the treatment, other drugs, compounds and/or materials used in combination with the particular fusion protein employed, the age, sex, weight, condition, general health and prior medical history of the cat being treated, and like factors well known in the medical arts.

In general, a suitable dose of a fusion protein will be that amount of the fusion protein that is the lowest dose effective to produce a therapeutic effect. Such an effective dose will generally depend upon the factors described above. Generally, intravenous, intramuscular and subcutaneous doses of the fusion protein for a cat will range from about 0.001 to about 1 milligrams (mg) per kilogram of body weight per day, e.g., 0.001-10 mg/kg/day, about 0.001-1 mg/kg/day, or about 0.003-0.30 mg/kg/day. In some embodiments, the fusion protein is administered at a dose greater than or equal to 0.005 mg/kg/day (e.g., greater than 0.006, 0.007, 0.008, 0.009 or 0.010 mg/kg/day). In still other embodiments, the fusion protein is administered at a dose of about 0.01, 0.05 or 0.10 milligrams per kilogram of body weight per week, or 0.10 mg/kg/week. In still other embodiments, the fusion protein is administered bi-weekly (e.g., once every 14 days) at a dose of about 0.01, 0.05 or 0.10 milligrams per kilogram of body weight per 14 days, or 0.10 mg/kg/14 days.

The present disclosure contemplates formulation of the fusion protein in any of the aforementioned pharmaceutical compositions and preparations. Furthermore, the present disclosure contemplates administration via any of the foregoing routes of administration. One of skill in the art can select the appropriate formulation and route of administration based on the condition being treated and the overall health, age, and size of the patient being treated.

### EXAMPLES

The present technology is further illustrated by the following Examples, which should not be construed as limiting in any way.

### Example 1: Synthesis and Methods of Making a GLP-1-analog-Fc Fusion Protein in Transiently Transfected CHO Cells

GLP-1-analog-Fc fusion proteins were synthesized as follows. A gene sequence of interest was constructed using proprietary software (Curia, Belmont, CA) and was cloned into a high expression mammalian vector. CHO cells were seeded in a shake flask 24 hours before transfection and were grown using serum-free chemically defined media. A DNA expression construct that encodes the GLP-1-analog-Fc fusion protein of interest was transiently transfected into a suspension of CHO cells using the (Curia, Belmont, CA) standard operating procedure for transient transfection. After 20 hours, the cells were counted to determine the viability and viable cell count, and the titer was measured by ForteBio® Octet® (Pall ForteBio LLC, Fremont, CA). Additional readings were taken throughout the transient transfection production run. The culture was harvested on or after Day 14.

### Example 2: Synthesis and Methods of Making a GLP-1-analog-Fc Fusion Protein in CHO Cells

A CHO cell line is originally derived from CHO-K1 (Curia, Belmont, CA), and the endogenous glutamine synthetase (GS) genes are knocked out by recombinant technology using methods known in the art. Stable expression DNA vectors are designed and optimized for CHO expression and GS selection and incorporated into a high expression mammalian vector (Curia, Belmont, CA). The sequence of each completed construct is confirmed prior to initiating scale-up experiments. The suspension-adapted CHO cells are cultured in a humidified 5% CO2 incubator at 37° C. in a chemically defined media (CD OptiCHO; Invitrogen, Carlsbad, CA). No serum or other animal-derived products are used in culturing the CHO cells.

Approximately 80 million suspension-adapted CHO cells, growing in CD OptiCHO media during the exponential growth phase, are transfected by electroporation using MaxCyte® STX® system (MaxCyte, Inc., Gaithersburg, MD) with 80 g DNA to create a stable CHO cell line for each GLP-1-analog-Fc fusion protein (DNA construct contains the full-length sequence of the GLP-1-analog-Fc fusion protein). After twenty-four hours, the transfected cells are counted and placed under selection for stable integration of the GLP-1-analog-Fc fusion genes. The transfected cells are seeded into CD OptiCHO selection media containing between 0-100 M methionine sulfoximine (MSX) at a cell density of 0.5×10⁶ cells/mL in a shaker flask and incubated at 37° C. with 5% CO2. During a selection process, the cells are spun down and resuspended in fresh selection media every 2-3 days until the CHO stable pool recovers its growth rate and viability. The cell culture is monitored for growth and titer.

The cells are grown to 2.5×10⁶ cells per mL. At the time of harvest for cell banking, the viability is expected to be above 95%. The cells are then centrifuged, and the cell pellet is resuspended in the CD OptiCHO media with 7.5% dimethyl sulfoxide (DMSO) to a cell count of 15×10⁶ cells per mL per vial. Vials are cryopreserved for storage in liquid nitrogen.

A small-scale-up production is performed using the CHO cells as follows. The cells are scaled up for production in CD OptiCHO growth medium containing up to 100 μM MSX at 37° C. and fed every 2-4 days as needed, with CD OptiCHO growth medium supplemented with glucose and additional amino acids as necessary for approximately 14-21 days. The MSX concentration is optionally increased over time to exert additional selectivity for clones capable of yielding higher product titers.

The conditioned media supernatant harvested from the stable pool production run is clarified by centrifuge spinning. The protein is run over a Protein A (MabSelect, Cytiva, Marlborough, MA) column pre-equilibrated with binding buffer. Washing buffer is then passed through the column until the OD280 value (NanoDrop, Thermo Fisher Scientific) is measured to be at or near background levels. The GLP-1-analog-Fc fusion protein is eluted using a low pH buffer, elution fractions are collected, and the OD280 value of each fraction is recorded. Fractions containing the target GLP-1-analog-Fc fusion protein are pooled and optionally further filtered using a 0.2 m membrane filter.

The cell line is optionally further subcloned to monoclonality and optionally further selected for high titer GLP-1-analog-Fc-fusion protein-expressing clones using the method of limiting dilution, a method known to those skilled in the art. After obtaining a high titer, monoclonal GLP-1-analog-Fc fusion protein-expressing cell line, production of the GLP-1-analog-Fc fusion protein is accomplished as described above in growth medium without MSX, or optionally in growth medium containing MSX, to obtain a cell culture supernatant containing the recombinant, CHO-made, GLP-1-analog-Fc fusion protein.

### Example 3: Purification of a GLP-1-analog-Fc Fusion Protein Manufactured in CHO Cells

Purification of a GLP-1-analog-Fc fusion protein was performed as follows. Conditioned media supernatants containing the secreted GLP-1-analog-Fc fusion protein were harvested from the CHO production runs and were clarified by centrifugation. The supernatant containing the desired GLP-1-analog-Fc fusion protein was run over a Protein A column, washed, and eluted using a low pH gradient. Afterwards, the eluted fractions containing the desired protein were pooled and buffer exchanged into 100 mM HEPES, 100 mM NaCl, 50 mM NaOAc, pH 6.0 buffer. A final filtration step was performed using a 0.2 μm membrane filter. The final protein concentration was calculated from the solution optical density at 280 nm. Further optional purification by ion-exchange chromatography (e.g., using an anion exchange bead resin or a cation exchange bead resin), gel filtration chromatography, or other methods was performed, as necessary.

### Example 4: GLP-1-analog-Fc Fusion Protein Structure Confirmation by Non-Reducing and Reducing CE-SDS

CE-SDS analysis was performed in a LabChip® GXII (Perkin Elmer, Waltham, MA) on a solution of a purified GLP-1-analog-Fc fusion protein dissolved in 200 mM HEPES, 100 mM NaCl, 50 mM NaOAc, pH 7.0 buffer, and the electropherogram was plotted. Under non-reducing conditions, the sample was run against known molecular weight (MW) protein standards, and the eluting peak represented the ‘apparent’ MW of the GLP-1-analog-Fc fusion protein homodimer.

Under reducing conditions (e.g., using beta-mercaptoethanol to break disulfide bonds of the GLP-1-analog-Fc fusion homodimer), the apparent MW of the resulting GLP-1-analog-Fc fusion protein monomer was compared against half the molecular weight of the GLP-1-analog-Fe fusion protein homodimer as a way of determining that the structural purity of the GLP-1-analog-Fc fusion protein was likely to be correct.

The non-reducing and reducing main peaks found via CE-SDS analysis for GLP-1-analog-Fc fusion proteins synthesized in CHO cells are shown in Table 2, and 2× the apparent MW of the resulting GLP-1-analog-Fc fusion protein monomer was compared to the molecular weight of the GLP-1-analog-Fc fusion protein homodimer. The results in Table 2 illustrate that the structural conformations of the GLP-1-analog-Fc fusion proteins are likely to be correct.

TABLE 2
CE-SDS Non-Reducing and Reducing Main Peak for GLP-1-Fc fusion
protein homodimers synthesized in CHO cells

| Sequence | Non-reducing (kDa) Peak 1 | Reducing (kDa) Peak | $\frac{M {W}_{h o m o d i m e r}}{2 \times M {W}_{m o n o m e r}}$ |
|---|---|---|---|
| SEQ ID NO: 9 | 82.25 | 44.00 | 0.9 |
| SEQ ID NO: 11 | 81.50 | 44.37 | 0.9 |
| SEQ ID NO: 12 | 78.09 | 41.24 | 0.9 |
| SEQ ID NO: 16 | 66.41 | 37.44 | 0.9 |
| SEQ ID NO: 17 | 68.81 | 39.48 | 0.9 |
| SEQ ID NO: 19 | 67.41 | 39.16 | 0.9 |
| SEQ ID NO: 20 | 82.97 | 45.93 | 0.9 |

### Example 5: GLP-1-analog-Fe Fusion Protein Sequence Identification by LC-MS with Glycan Removal

To obtain an accurate estimate of the GLP-1-analog-Fc fusion protein mass via mass spectroscopy (MS), the sample is first treated to remove naturally occurring glycan that might interfere with the MS analysis. 100 L of a 2.5 mg/mL GLP-1-analog-Fc fusion protein dissolved in 100 mM HEPES, 100 mM NaCl, 50 mM NaOAc, pH 6.0 buffer solution is first buffer exchanged into 0.1 M Tris, pH 8.0 buffer containing 5 mM EDTA using a Zeba desalting column (Pierce, Thermo Fisher Scientific, Waltham, MA). 3.33 L of PNGase F enzyme (New England Biolabs Remove-iT® PNGase F) is added to this solution to remove N-linked glycan present in the fusion protein (e.g., glycan linked to the side chain of the asparagine located at the cNg-N site), and the mixture is incubated at 37° C. overnight in an incubator. The overnight incubated mixture is then buffer exchanged into Phosphate Buffered Saline. The sample is then analyzed via LC-MS (Novatia LLC, Newtown, PA) resulting in a molecular mass of the molecule which corresponds to the desired homodimer without the glycan. This mass is then further corrected since the enzymatic process used to cleave the glycan from the cNg-asparagine also deaminates the asparagine side chain to form an aspartic acid, and in doing so the enzymatically treated homodimer gains 2 Da overall, corresponding to a mass of 1 Da for each chain present in the homodimer. Therefore, the actual molecular mass is the measured mass minus 2 Da to correct for each of the enzymatic modifications of the GLP-1-analog-Fc fusion protein structure in the analytical sample.

### Example 6: % Homodimer by Size-Exclusion Chromatography

Size-exclusion chromatography (SEC-HPLC) of GLP-1-analog-Fc fusion proteins was carried out using a Waters 2795 HPLC (Waters Corporation, Milford, MA) connected to a 2998 Photodiode array at a wavelength of 280 nm. 100 μL or less of a sample containing a GLP-1-analog-Fc fusion protein of interest was injected into an AdvanceBio SEC 300 Å, 4.6×300 mm, 2.7 μm, LC column (Agilent Technologies, Santa Clara, CA) operating at a flow rate of 0.2 mL/min and with a mobile phase comprising 50 mM sodium phosphate, 300 mM NaCl, and 0.05% w/v sodium azide, pH 6.2. The AdvanceBio SEC column operates on the principle of molecular size separation. Therefore, larger soluble GLP-1-analog-Fc fusion protein aggregates (e.g., multimers of GLP-1-analog-Fc fusion protein homodimers) elute at earlier retention times, and the non-aggregated homodimers elute at later retention times. In separating the mixture of homodimers from aggregated multimeric homodimers via analytical SEC-HPLC, the purity of the GLP-1-analog-Fc fusion protein solution in terms of the percentage of non-aggregated homodimer was ascertained.

### Example 7: In Vitro Fc (Gamma) Receptor Binding Affinity for a GLP-1-Analog-Fc Fusion Protein

The binding of a GLP-1-analog-Fc fusion protein to Fc(gamma) receptors at pH 7.4 is conducted using an ELISA assay as follows. Human Fc(gamma) receptors I, IIa, IIb, and III are used as mammalian receptors. A GLP-1-analog-Fc fusion protein is diluted to 10 μg/mL in sodium bicarbonate buffer at pH 9.6 and coated on Maxisorp (Nunc) microtiter plates overnight at 4° C., after which the microplate strips are washed 5 times with wash (PBS/0.05% Tween-20) buffer and blocked with Superblock blocking reagent (Thermo Fisher Scientific). Serial dilutions of His-tagged rhFc(gamma) receptors (recombinant human Fc(gamma)RI, Fc(gamma)RIIa, Fc(gamma)RIIb, or Fc(gamma)RIII; AcroBiosystems) are prepared in PBST/10% (PBS/0.1% Tween-20/10% SuperBlock) Superblock buffer from 6000 ng/mL to 8.2 ng/mL and loaded at 100 μL/well onto the microplate strips coated with the GLP-1-analog-Fc fusion protein. The microtiter plate is incubated for 1 hour at room temperature (22° C.) after which the microplate strips are washed 5 times with wash buffer and then loaded with 100 μL/well of streptavidin-HRP diluted 1:10000 in PBST/10% Superblock buffer. After incubating for 1 hour at 22° C., the microplate strips are washed again 5 times with wash buffer. Trimethylbenzidine (TMB) is added to reveal the bound Fc(gamma) receptor proteins and stopped with ELISA stop reagent (Boston Bioproducts). The plate is read in an ELISA plate reader at 450 nm, and the OD values (proportional to the binding of each rhFc(gamma) receptor to the GLP-1-analog-Fc fusion protein) are plotted against log concentrations of each rhFc(gamma) receptor added to each well to generate binding curves using GraphPad Prism software.

### Example 8: In Vitro ELISA Assay for Evaluating GLP-1 Receptor Binding Affinity of a GLP-1-analog-Fc Fusion Protein

The GLP-1-analog-Fc fusion protein binding affinity towards the GLP-1 receptor was determined by an enzyme-linked immunosorbent assay (ELISA). The GLP-1-analog-Fc fusion protein was diluted in PBST/SB (PBS/0.1% Tween-20/10% SuperBlock) from 2000 ng/mL to 0.914 ng/mL and then added to microtiter plate wells previously coated with Recombinant Human GLP-1 Receptor protein containing a human Fc tag (Sino Biologicals, Catalog #13944-H02H) in carbonate buffer, blocked with SuperBlock™ (Thermo Fisher Scientific) and then incubated with diluted compounds for one hour. After washing the microtiter plate with wash buffer (PBS/0.05% Tween-20) to remove all unbound compounds, the compounds bound to the plate wells were detected by incubating the plate wells with Goat anti-cat IgG-Fc Fragment Antibody conjugated to HRP enzyme (Bethyl Laboratory, Catalog #A20-117P) diluted between 1:10000 and 1:40000 in PBST/SB buffer for one hour. Following wash with wash buffer and purified water, trimethylbenzidine (TMB) reagent was added to each well which was catalyzed by the HRP enzyme and incubated for 6 minutes. This caused a colorimetric change that was proportional to the amount of bound HRP-antibody conjugate. The enzyme substrate reaction was then stopped by the addition of Stop Reagent (1% H2SO4) and the color intensity (optical density, OD) of each well was measured using a spectrophotometric microplate reader at 450 nm wavelength. The OD450 values were further converted using a 4-parameter regression curve fit algorithm in SoftMax Pro software (Molecular Devices).

### Example 9: In Vitro ELISA Assay for Evaluating GLP-1-Analog-Fc Fusion Protein Levels in Feline Serum

GLP-1-analog-Fc fusion protein (compound) levels in feline serum were determined by an ELISA. The serum samples were diluted in Sample Dilution Buffer (SDB; contained a mixture of SuperBlock™ (Thermo Fisher Scientific) in PBS/0.1% Tween-20 and 20% Horse Serum) at 1:10-100 and were then added to microtiter plate wells previously coated with recombinant monoclonal rabbit anti-human GLP-1 antibody (R&D Systems, Catalog #MAB12490-100) protein in carbonate buffer, blocked with SuperBlock™ (Thermo Fisher Scientific) and then incubated with diluted serum test samples for one hour at room temperature (22° C.). Soluble standards, prepared from purified compounds via serial dilutions in PBST/SB/20% HS/EDTA/5% cat serum buffer, were added to microtiter plate wells, previously coated with recombinant monoclonal rabbit anti-human GLP-1 antibody and blocked with SuperBlock™, and were incubated for one hour at room temperature (22° C.). After washing the plate with wash buffer (PBS/0.05% Tween-20) to remove all unbound compounds, the compounds bound to the plate (including soluble standards containing compounds) were detected by incubating microtiter plate wells with Goat anti-cat IgG-Fc Fragment Antibody conjugated to HRP enzyme (Bethyl Laboratory, Catalog #A20-117P) diluted between 1:10000 and 1:40000 in PBST/SB/20% HS/EDTA buffer for 45 minutes. Following washing with PBST buffer and purified water, trimethylbenzidine (TMB) reagent was added to each well which was catalyzed by the HRP enzyme and was incubated for 12 minutes. This causes a colorimetric change that was proportional to the amount of bound HRP-antibody conjugate. The enzyme substrate reaction was then stopped by the addition of Stop Reagent (1% H2SO4) and the color intensity (optical density, OD) of each well was measured using a spectrophotometric microplate reader at 450 nm wavelength. The OD450 values were further converted using a 4-parameter regression curve fit algorithm in SoftMax Pro software (Molecular Devices).

### Example 10: In Vivo Evaluation of GLP-1-Analog-Fc Fusion Protein Pharmacokinetic Parameters in Domestic Felines

Groups of cats (N=3; (Transpharmation Canada Ltd., Fergus, Ontario)) of mixed ages (8 months to 1 year of age) were injected subcutaneously (s.c.) on Day 0 with 0.10 mg/kg of a GLP-1-analog-Fc fusion protein. In some examples, cats were given an additional subcutaneous (s.c.) dose at 51 days post the initial dose with the same GLP-1-analog-Fc fusion protein at a dose of 0.10 mg/kg. All cats were non-terminally bled via jugular venipuncture before dosing (Day 0) as well as 4, 8, and 12 hours post-dose. Additional samples were collected on Days 1, 2, 3, 4, 5, 6, 7, 8, 10, 12, and 14. For cats that received a second dose, additional blood collections were performed on Days 0, 7, 14, and 28 post-second dose. Blood samples were allowed to clot and were centrifuged to obtain serum samples for the quantification of the GLP-1-analog-Fc fusion protein concentration by ELISA.

### Example 11: In Vitro ELISA Assay for Evaluating GLP-1-Analog-Fc Fusion Protein Levels in Feline Serum

GLP-1-analog-Fc fusion protein (compound) levels in feline serum are determined by an ELISA. The serum samples are diluted in Sample Dilution Buffer (SDB; contains a mixture of SuperBlock™ (Thermo Fisher Scientific) in PBS/Tween and 20% Horse Serum) at 1:10-100 and are then added to microtiter plate wells previously coated with recombinant monoclonal rabbit anti-human GLP-1 antibody (R&D Systems, Catalog #MAB12490-100) protein in carbonate buffer, blocked with SuperBlock™ (Thermo Fisher Scientific) and then incubated with diluted serum test samples for one hour. Soluble standards, prepared from purified compounds via serial dilutions in PBST/SB/20% HS/EDTA/5% cat serum buffer, are added to microtiter plate wells, previously coated with recombinant monoclonal rabbit anti-human GLP-1 antibody and blocked with SuperBlock™, and are incubated for one hour. After washing the plate with wash buffer (PBS/Tween; PBST) to remove all unbound compounds, the compounds bound to the plate (including soluble standards containing compounds) are detected by incubating microtiter plate wells with Goat anti-cat IgG-Fc Fragment Antibody conjugated to HRP enzyme (Bethyl Laboratory, Catalog #A20-117P) diluted between 1:10000 and 1:40000 in PBST/SB/20% HS/EDTA buffer for 45 minutes. Following washing with PBST buffer and purified water, trimethylbenzidine (TMB) reagent is added to each well which is catalyzed by the HRP enzyme and is incubated for 12 minutes. This causes a colorimetric change that is proportional to the amount of bound HRP-antibody conjugate. The enzyme substrate reaction is then stopped by the addition of Stop Reagent (1% H2SO4) and the color intensity (optical density, OD) of each well is measured using a spectrophotometric microplate reader at 450 nm wavelength. The OD450 values are further converted to antibody titers (e.g., levels) using the mouse IgG standards, using a 4-parameter regression curve fit algorithm in SoftMax Pro software (Molecular Devices).

### Example 12: In Vivo Evaluation of GLP-1-analog-Fc Fusion Protein Pharmacokinetic Parameters in Domestic Felines

Groups of cats (N=3; (Transpharmation Canada Ltd., Fergus, Ontario)) of mixed ages (8 months to 1 year of age) are injected subcutaneously (s.c.) on Day 0 with 0.10 mg/kg of a GLP-1-analog-Fc fusion protein. In some examples, cats are given an additional subcutaneous (s.c.) dose at 51 days post the initial dose with the same GLP-1-analog-Fc fusion protein at a dose of 0.10 mg/kg. All cats are non-terminally bled via jugular venipuncture before dosing (Day 0) as well as 4, 8, and 12 hours post-dose. Additional samples are collected on Days 1, 2, 3, 4, 5, 6, 7, 8, 10, 12, and 14. For cats that receive a second dose, additional blood collections are performed on Days 0, 7, 14, and 28 post-second dose. Blood samples are allowed to clot and are centrifuged to obtain serum samples for the quantification of the GLP-1-analog-Fc fusion protein concentration by ELISA.

### Example 13: In Vitro ELISA Assay for Evaluating Anti-Drug Antibody Levels in Cat Serum

Serum anti-drug antibody titers are determined by enzyme-linked immunosorbent assay (ELISA). Serum samples are diluted in Sample Dilution Buffer (SDB; contains a mixture of 10% Superblock (Thermo Fisher Scientific) in PBST/EDTA and 20% Horse Serum) at 1:100-10000 and then added to microtiter plate wells previously coated with GLP-1-analog-Fc fusion protein (the compound) (Akston Biosciences) in carbonate buffer, blocked with SuperBlock™ (Thermo Fisher Scientific) and then incubated with diluted serum test samples for one hour. Cat IgG antibody (Jackson Immunoresearch, Catalog #002-000-003) in carbonate buffer is directly coated to microplate wells with the cat IgG antibody pre-diluted via serial dilutions to create a standard curve that allows for quantitation of antibody (Ab) titers in the serum test samples. After washing the plate with wash buffer (PBS/0.05% Tween 20; PBST) to remove all unbound serum proteins, the bound GLP-1 specific cat IgG Abs bound to the plate (including precoated cat IgG standards or cat serum containing anti-GLP-1 Abs bound to the compound-coated microplate) are detected by incubating microplate wells with goat anti-cat-IgG F(ab′)2 conjugated to HRP (Jackson Immunoresearch, Catalog #102-035-006) diluted between 1:4000 and 1:30000 in PBS/0.05% Tween-20/10% SuperBlock™ buffer for 1 hour. Following washes with PBST buffer and purified water, trimethylbenzidine (TMB) reagent is added to each well which is catalyzed by the HRP enzyme and incubated for 5-20 minutes. This causes a colorimetric change that is proportional to the amount of bound HRP-antibody conjugate. The enzyme substrate reaction is then stopped by the addition of Stop Reagent (1% H2SO4) and the color intensity (optical density, OD) of each well is measured using a spectrophotometric microplate reader at 450 nm wavelength. The OD450 values are further converted to antibody titers (e.g., levels) using the cat IgG standards, using a 4-parameter regression curve fit algorithm in SoftMax Pro software (Molecular Devices).

### Example 14: GLP-1-analog-Fc Fusion Protein Sequence Identification by LC-MS with Glycan Removal

To obtain an accurate estimate of the GLP-1-analog-Fc fusion protein mass via mass spectroscopy (MS), the sample was first treated to remove naturally occurring glycan that might interfere with the MS analysis. 100 μL of a 2.5 mg/mL GLP-1-analog-Fc fusion protein dissolved in 100 mM HEPES, 100 mM NaCl, 50 mM NaOAc, pH 6.0 buffer solution was first buffer exchanged into 0.1 M Tris, pH 8.0 buffer containing 5 mM EDTA using a Zeba desalting column (Pierce, Thermo Fisher Scientific, Waltham, MA). 3.33 μL of PNGase F enzyme (New England Biolabs Remove-iT® PNGase F) was added to this solution to remove N-linked glycan present in the fusion protein (e.g., glycan linked to the side chain of the asparagine located at the cNg-N site), and the mixture was incubated at 37° C. overnight in an incubator. The overnight incubated mixture was then buffer exchanged into Phosphate Buffered Saline. The sample was then analyzed via LC-MS (Novatia LLC, Newtown, PA) resulting in a molecular mass of the molecule which corresponded to the desired homodimer without the glycan. This mass was then further corrected since the enzymatic process used to cleave the glycan from the cNg-asparagine also deaminated the asparagine side chain to form an aspartic acid, and in doing so the enzymatically treated homodimer gained 2 Da overall, corresponding to a mass of 1 Da for each chain present in the homodimer. Therefore, the actual molecular mass was the measured mass minus 2 Da to correct for each of the enzymatic modifications of the GLP-1-analog-Fc fusion protein structure in the analytical sample.

### Example 15: In Vivo Evaluation of Once-a-Week Injectable GLP-1-Analog-Fc Fusion Protein Candidates for Minimizing Weight Gain in Non-Obese Laboratory Cats

Groups of domestic cats (N=4 per group; Marshall BioResources, North Rose, New York), each over one year of age and of mixed ages, were administered subcutaneous (s.c.) injections once weekly for a period of 12 weeks with a dosing washout period over an additional 4 weeks for a 16-week total study duration. Each animal received between 0.10-0.70 mg/kg of a GLP-1-analog-Fc fusion protein per dose once-weekly. Non-terminal blood collection was performed via cephalic or saphenous venipuncture prior to the initial dose (Week 1), with subsequent weekly sampling conducted from Weeks 5 through 16. Blood samples were allowed to clot and were centrifuged to isolate serum, which was used to quantify circulating GLP-1-analog-Fc fusion protein concentrations via enzyme-linked immunosorbent assay (ELISA) following Example 9.

Food consumption was monitored by delivering a defined daily ration once each morning at approximately 8:00 AM±1 hour. Prior to feeding, residual food from the previous day was collected, weighed, and recorded. Notably, the animals did not disperse food throughout the housing environment, thereby allowing for accurate quantification of uneaten food retained within the feeding bowls. All weight measurements were performed using an OHAUS VALOR™ 1000 food scale, calibrated weekly using a certified 100 g calibration weight. On study days requiring fasted blood collection, food was removed from each cage at 4:00 PM±1 hour on the preceding afternoon, and the quantity removed was weighed and documented.

Body weight was measured weekly prior to compound administration and expressed as a percentage of each subject's baseline (Day 0) weight to normalize inter-animal variability. Group mean values and standard error of the mean (SEM) were calculated for each time point. To assess treatment-related effects on body weight, a paired t-test was performed at each study week, comparing the mean percent change in body weight of each treatment group to the corresponding vehicle control group. The use of a paired t-test allowed for statistical evaluation of within-subject changes over time and enabled identification of statistically significant differences attributable to treatment.

All statistical analyses were conducted using GraphPad Prism (version 10.4.0) or functionally equivalent statistical analysis software.

### Example 16: In Vivo Evaluation of a Once-a-Week Injectable GLP-1-Analog-Fc Fusion Protein Candidates for Minimizing Weight Gain in Non-Obese Laboratory Cats

Groups of domestic cats (N=4 per group), each over one year of age and of mixed ages, are administered subcutaneous (s.c.) injections once weekly for a period of 12 weeks plus a four-week dosing washout period for a total study duration of 16 weeks. Each animal receives between 0.10 to 0.70 mg/kg of a GLP-1-analog-Fc fusion protein per dose once-weekly. Non-terminal blood collection is performed via cephalic or saphenous venipuncture prior to the initial dose (Week 1), with subsequent weekly sampling conducted from Weeks 5 through 16. Blood samples are allowed to clot and are centrifuged to isolate serum, which is used to quantify circulating GLP-1-analog-Fc fusion protein concentrations via enzyme-linked immunosorbent assay (ELISA) following Example 9.

Food consumption is monitored by delivering a defined daily ration once each morning at approximately 8:00 AM±1 hour. Prior to feeding, residual food from the previous day is collected, weighed, and recorded. Notably, the animals do not disperse food throughout the housing environment, thereby allowing for accurate quantification of uneaten food retained within the feeding bowls. All weight measurements are performed using an OHAUS VALOR™ 1000 food scale, calibrated weekly using a certified 100 g calibration weight. On study days requiring fasted blood collection, food is removed from each cage at 4:00 PM±1 hour on the preceding afternoon, and the quantity removed is weighed and documented.

Body weight is measured weekly prior to compound administration and expressed as a percentage of each subject's baseline (Day 0) weight to normalize inter-animal variability. Group mean values and standard error of the mean (SEM) are calculated for each time point. To assess treatment-related effects on body weight, a paired t-test is performed at each study week, comparing the mean percent change in body weight of each treatment group to the corresponding vehicle control group. The use of a paired t-test allows for statistical evaluation of within-subject changes over time and enables identification of statistically significant differences attributable to treatment.

All statistical analyses are conducted using GraphPad Prism or functionally equivalent statistical analysis software.

### EQUIVALENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. In general, where the disclosure, or aspects of the disclosure, is/are referred to as comprising elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprise(s),” “comprising,” “contain(s),” and “containing” are intended to be open and the use thereof permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Additional advantages of the various embodiments of the technology will be apparent to those skilled in the art upon review of the disclosure herein and the working examples. It will be appreciated that the various embodiments described herein are not necessarily mutually exclusive unless otherwise indicated herein. For example, a feature described or depicted in one embodiment may also be included in other embodiments but is not necessarily included. Thus, the present invention encompasses a variety of combinations and/or integrations of the specific embodiments described herein.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

We claim:

1. A fusion protein comprising a Glucagon Like Peptide 1 (GLP-1)-analog, wherein the fusion protein comprises the following sequence:
(SEQ ID NO: 19)
QHVFLAPQQALSLLQRVRRHGEGTFTSDVSSYLEEQAAKEFIAWLVKGGG
GGGGSGGGGSGGGGSDCPKCPPPEMLGGPSIFIFPPKPKDTLSISRTPEV
TCLVVDLGPDDSDVQITWFVDNTQVYTAKTSPREEQFQSTYRVVSVLPIL
HQDWLKGKEFKCKVNSKSLPSPIERTISKDKGQPHEPQVYVLPPAQEELS
RNKVSVTCLIEGFYPSDIAVEWEITGQPEPENNYRTTPPQLDSDGTYFLY
SRLSVDRSRWQRGNTYTCSVSHEALHSHHTQKSLTQSPG.

2. The fusion protein of claim 1, wherein the fusion protein is a homodimer comprising two identical monomers bound together via one or more disulfide bonds.

3. The fusion protein of claim 1, said fusion protein consisting of SEQ ID NO: 19.

4. A composition comprising a fusion protein according to claim 1 and a pharmaceutically acceptable carrier.

5. The composition of claim 4, said composition comprising homodimers of said fusion protein.

6. The composition of claim 5, wherein the percentage homodimers of said fusion protein is greater than or equal to 85%.

7. A method for treatment of obesity, prevention of obesity, or treatment of conditions associated with excess weight in cats, wherein the method comprises administering a therapeutically effective amount of a fusion protein according to claim 1 or composition thereof to a cat in need thereof.

8. The method of claim 7, wherein the fusion protein or composition thereof is administered via injection.

9. The method of claim 7, wherein the fusion protein or composition thereof is administered subcutaneously or intramuscularly.

10. The method of claim 7, wherein the fusion protein has a measurable serum pharmacokinetic half-life in said cat of longer than about 1 day, 1.5 days, 2 days, 2.2 days, 2.5 days, 3 days or longer, after said fusion protein or composition thereof is administered.

11. The method of claim 7, wherein said cat exhibits reduced food intake over a period of 3 days, 4 days, 5 days, 6 days, 7 days, or longer, after said fusion protein or composition thereof is administered.

12. A method of producing the fusion protein of claim 1, said method comprising transiently transfecting a nucleic acid encoding for the fusion protein into a Chinese hamster ovary (CHO) cell, wherein the transfected CHO cell expresses the fusion protein, and wherein the yield of the purified or isolated fusion protein is greater than 300 mg/L.

13. A cell engineered to express the fusion protein of claim 1.

14. The cell of claim 13, wherein said cell is a Chinese hamster ovary (CHO) cell.

15. A cDNA encoding the fusion protein of claim 1.
